(19) United States
(12) Patent Application Publication
(10) Pub. No.: US 2018/0221312 A1
(43) Pub. Date: Aug. 9, 2018

(54) CXCR-2 INHIBITORS FOR TREATING DISORDERS

(71) Applicant: ARDEA BIOSCIENCES, INC., San Diego, CA (US)

(72) Inventors: Payal NANAVATI, San Diego, CA (US); Johan HOEGSTEDT, San Diego, CA (US); Jesse HALL, San Diego, CA (US)

(21) Appl. No.: 15/702,693

(22) Filed: Sep. 12, 2017

Related U.S. Application Data
(63) Continuation-in-part of application No. 15/516,465, filed on Apr. 3, 2017.
(60) Provisional application No. 62/307,348, filed on Mar. 11, 2016.

(51) Int. Cl. A61K 31/165; A61K 31/506; A61P 25/28; A61P 37/06

(52) U.S. Cl. CPC A61K 31/165; A61P 37/06; A61P 25/28; A61K 31/506

(57) ABSTRACT

N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), a known chemokine modulator, in combination with colchicine is useful in the treatment of diseases/conditions in which modulation of chemokine receptor activity, interleukin-1 (IL-1) activity, and/or myeloperoxidase (MPO) activity is beneficial. In particular, provided herein are compositions and methods for the treatment and prevention of such diseases/conditions.

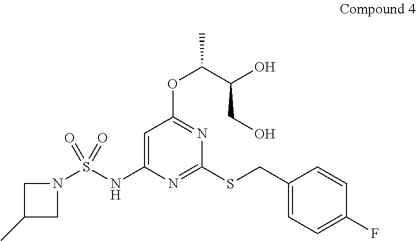 [SMILES: CC1CN(S(=O)(=O)Nc2cc(O[C@H](C)[C@@H](O)CO)nc(SCc3ccc(F)cc3)n2)C1]

### CROSS-REFERENCE TO RELATED APPLICATIONS

This application is a continuation-in-part of U.S. Ser. No. 15/516,465, filed Apr. 3, 2017, which is a 35 U.S.C. § 371 National Stage Application of PCT Application No. PCT/US2017/021570, filed Mar. 9, 2017, which claims the benefit of U.S. Provisional Application Ser. No. 62/307,348, filed Mar. 11, 2016. The disclosure of each of the prior applications is considered part of, and is hereby incorporated by reference in its entirety, in the disclosure of this application.

### BACKGROUND OF THE INVENTION

Chemokines play an important role in immune and inflammatory responses in various diseases and disorders. These small secreted molecules are a growing superfamily of 8-14 kDa proteins characterized by a conserved cysteine motif. The chemokine superfamily comprises three groups exhibiting characteristic structural motifs, the C—X—C, C—C and C—X₃—C families. The C—X—C chemokines include several potent chemo-attractants and activators of neutrophils.

### SUMMARY OF THE INVENTION

Provided herein are CXCR-2 inhibitor compounds and pharmaceutical compositions comprising said compounds. Some embodiments provided herein describe a method for treating or preventing a disease or disorder in a subject, comprising administering to the subject a combination of: (i) Colchicine, or a pharmaceutically acceptable salt thereof; and (ii) a CXCR-2 inhibitor, or a pharmaceutically acceptable salt thereof. In some embodiments, the combination is a synergistic combination. In further or additional embodiments, the disease or disorder is an inflammation-mediated disease or disorder, an autoimmune disease or disorder, a neutrophil-mediated disease or disorder, or a hematopoietic disease or disorder.

Some embodiments provided herein describe a method for treating or preventing an interleukin-1 (IL-1)- or myeloperoxidase (MPO)-mediated disease or disorder in a subject in need thereof, comprising administering to the subject, a therapeutically effective amount of a combination of: (i) Colchicine, or a pharmaceutically acceptable salt thereof; and (ii) a CXCR-2 inhibitor, or a pharmaceutically acceptable salt thereof. In some embodiments, the combination is a synergistic combination. In certain specific embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide or a pharmaceutically acceptable salt thereof.

In some embodiments, the IL-1-mediated disease or disorder is an inflammation-mediated disease or disorder, an autoimmune disease or disorder, neutrophil-mediated disease or disorder, or a hematopoietic disease or disorder. In certain embodiments, the inflammation-mediated disease or disorder is a vascular or cardiovascular disease or disorder, a neuroinflammatory disease or disorder, a dermatological disease or skin disorder, pancreatitis, or an inflammatory or allergic disease of the airways.

In some embodiments, the vascular or cardiovascular disease or disorder is coronary artery disease, coronary heart disease, ischemic heart disease, peripheral arterial disease, cerebrovascular disease, stroke, renal artery stenosis, aortic aneurysm, cardiomyopathy, hypertensive heart disease, high blood pressure, hypertension, heart failure, pulmonary heart disease, cardiac dysrhythmias, abnormalities of heart rhythm, inflammatory heart disease, endocarditis, inflammatory cardiomegaly, myocarditis, valvular heart disease, congenital heart disease, rheumatic heart disease, re-perfusion injury, or atherosclerosis, or any combination thereof.

In certain embodiments, the neuroinflammatory disease or disorder is Alzheimer's Disease.

In some embodiments, the pancreatitis is acute pancreatitis, chronic pancreatitis, alcohol induced pancreatitis, gallstone induced pancreatitis, drug induced pancreatitis, auto-immune pancreatitis, procedure induced pancreatitis, or trauma induced pancreatitis, or any combination thereof.

In some embodiments, the dermatological disease or skin disorder is rosacea, eczema, acne, hidradenitis suppurativa, Palmo-Plantar Pustulosis, Generalized Pustular Psoriasis, Pyoderma Gangrenosum, Erosive Pustular Dermatosis of the Scalp, Sweet's Syndrome, Bowel-associated Dermatosis-arthritis Syndrome, Pustular Psoriasis, Acute Generalized Exanthematous Pustulosis, Keratoderma Blenorrhagicum, Sneddon-Wilkinson Disease, IgA Pemphigus, Amicrobial Pustulosis of the Folds, Infantile Acropustulosis, Transient Neonatal Pustulosis, Neutrophilic Eccrine Hidradenitis, Rheumatoid Neutrophilic Dermatitis, Neutrophilic Urticaria, Dermatitis Herpetiformis, Linear IgA disease (LAD), Inflammatory Epidermolysis Bullosa Aquisita, Alopecia Areata, Autoimmune Angioedema, Autoimmune progesterone dermatitis, Autoimmune urticaria, Bullous pemphigoid, Cicatricial pemphigoid, Dermatitis herpetiformis, Epidermolysis bullosa acquisita, Erythema nodosum, Gestational pemphigoid, Lichen planus, Lichen sclerosus, Morphea, Pemphigus vulgaris, Pityriasis lichenoides et varioliformis acuta, Mucha-Habermann disease, Vitiligo, or Neutrophilic Dermatosis of the Dorsal Hands, or any combination thereof.

In some embodiments, the autoimmune disease or disorder is Pustular Vasculitis, Small Vessel Vasculitis, Urticarial Vasculitis, Autoimmune urticaria, Medium Vessel Vasculitis, rheumatoid arthritis, Celiac disease, Graves' disease, Sjorgen syndrome, scleroderma, thyroiditis, myasthenia gravis, vasculitis, Addison's disease, autoimmune hepatitis, myocarditis, postmyocardial infarction syndrome, postpericardiotomy syndrome, subacute bacterial endocardititis, Anti-Glomerular Basement Membrane nephritis, Interstitial cystitis, lupus nephritis, systemic lupus, bullous systemic lupus erythmatosus, Primary biliary cirrhosis (PBC), Primary sclerosing cholangitis, Antisynthetase syndrome, Ord's thyroiditis, Autoimmune Oophoritis, Autoimmune orchitis, Autoimmune enteropathy, Chron's disease, microscopic colitis, ulcerative colitis, Antiphospholipid syndrome (APS), Aplastic anemia, Autoimmune hemolytic anemia, Autoimmune lymphoproliferative syndrome, Autoimmune neutropenia, or Autoimmune thrombocytopenic purpura.

In certain embodiments, the hematopoietic disease or disorder is an anemia, a blood coagulation disorder, a blood platelet disorder, a blood protein disorder, erythroblastosis, hematologic neoplasm, hemoglobinopathies, a hemorrhagic disorder, a leukocyte disorder, methemoglobinemia, pancytopenia, polycythemia, preleukemia, sulfhemoglobinemia, or thrombophilia.

In other embodiments, the IL-1-mediated disease or disorder is a disease of the respiratory tract, a disease of the bones and/or joints, a skin disease, a disease of the gastrointestinal tract, a disease of central and/or peripheral nervous system, cancer, cystic fibrosis, a burn wound, a chronic skin ulcer, a reproductive disease, a re-perfusion injury, allograft rejection, atherosclerosis, Acquired Immunodeficiency Syndrome (AIDS), lupus erythematosus, systemic lupus erythematosus, Hashimoto's thyroiditis, diabetes mellitus type I, diabetes mellitus type II, nephrotic syndrome, eosinophilia fascitis, hyper IgE syndrome, lepromatous leprosy, and idiopathic thrombocytopenia pupura, post-operative adhesions, sepsis, septic shock, Behcet's Disease, Still's Disease, Erythema Marginatum, Unclassified Periodic Fever Syndromes, Autoinflammatory Syndromes, or Erythema Elevatum Diutinum, or any combination thereof.

In some embodiments, the inflammatory or allergic disease of the airways is chronic bronchitis, chronic obstructive bronchitis (COPD), asthma, bronchiectasis, allergic or non-allergic rhinitis or sinusitis, cystic fibrosis, α-1-antitrypsin deficiency, coughs, pulmonary emphysema, pulmonary fibrosis, idiopathic pulmonary fibrosis, or hyper-reactive airways, or any combination thereof.

In other embodiments, the MPO-mediated disease or disorder is acute myeloid leukemia (AML), chronic myeloid leukemia (CML), polycythemia vera, Hodgkin disease, refractory megaloblastic anemia, aplastic anemia, myelofibrosis with myeloid metaplasia, myelodysplastic syndromes, acute coronary syndrome (ACS), cardiovascular disease, kidney disease, chronic obstructive pulmonary disease (COPD), Alzheimer's disease, inflammatory bowel disease, atherosclerotic disease, or rheumatoid arthritis (RA).

Other embodiments provided herein describe a method for treating an inflammation-mediated disease or disorder or a neutrophil-mediated disease or disorder in a subject in need thereof, comprising administering to the subject a therapeutically effective amount of a combination of: (i) Colchicine, or a pharmaceutically acceptable salt thereof; and (ii) a CXCR-2 inhibitor, or a pharmaceutically acceptable salt thereof. In some embodiments, the combination is a synergistic combination. In certain specific embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide or a pharmaceutically acceptable salt thereof. In some embodiments, the inflammation-mediated disease or disorder is related to elevated CXCR-2 levels.

Also described herein in some embodiments is a method for reducing the level of biomarkers of acute inflammation in a subject in need thereof, comprising administering to the subject a therapeutically effective amount of a combination of: (i) Colchicine, or a pharmaceutically acceptable salt thereof, and (ii) a CXCR-2 inhibitor, or a pharmaceutically acceptable salt thereof. In certain specific embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide or a pharmaceutically acceptable salt thereof. In some embodiments, the acute inflammation biomarker is Insulin, Myeloperoxidase, Microalbumin, C-reactive protein, Osteopontin, glutathione S transferase α, IL-1β, TNF-α, IL-6, IL-8, SAA, VCAM-1, ICAM-1, NGAL, KIM1, MCP-1, or any combination thereof. In certain specific embodiments, the acute inflammation biomarker is myeloperoxidase, IL-1β, or a combination thereof.

Other embodiments provided herein describe a method of treating or preventing a chemokine-mediated disease or disorder in a subject in need thereof, comprising administering to the subject a therapeutically effective amount of a synergistic combination of: (i) Colchicine, or a pharmaceutically acceptable salt thereof; and (ii) a CXCR-2 inhibitor, or a pharmaceutically acceptable salt thereof. In certain specific embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide or a pharmaceutically acceptable salt thereof. In some embodiments, the chemokine mediated disease or disorder is arthritis, chronic obstructive pulmonary disease, adult or acute respiratory distress syndrome, asthma, atherosclerosis, myocardial and renal ischemia/reperfusion injury, peripheral limb ischemia/reperfusion injury, inflammatory bowel disease, ulcerative colitis, Crohn's disease, meconium apriration syndrome, atopic dermatitis, cystic fibrosis, psoriasis, psoriatic arthritis, multiple sclerosis, angiogenesis, restenosis, osteoarthritis, osteoporosis, septic shock, endotoxic shock, gram negative sepsis, toxic shock syndrome, stroke, glomerulonephritis, thrombosis, graft vs. host reaction, allograft rejections, transplant reperfusion injury, early transplantation rejection, acute inflammation, alzheimers disease, malaria, respiratory viruses, herpes viruses, hepatitis viruses, HIV, Kaposi's sarcoma-associated viruses, meningitis, gingivitis, herpes encephalitis, CNS vasculitis, traumatic brain injury, brain ischemia/reperfusion injury, migraine, CNS tumors, subarachnoid hemorrhage, post surgical trauma, interstitial pneumonitis, hypersensitivity, crystal induced arthritis, acute and chronic pancreatitis, hepatic ischemia/reperfusion injury, acute alcoholic hepatitis, necrotizing enterocolitis, chronic sinusitis, uveitis, polymyositis, vasculitis, acne, gastric and duodenal ulcers, intestinal ischemia/reperfusion injury, celiac disease, esophagitis, glossitis, rhinitis, airflow obstruction, airway hyperresponsiveness, bronchiolitis, bronchiolitis obliterans, bronchiolitis obliterans organizing pneumonia, bronchiectasis, chronic bronchitis, cor pulmonae, dyspnea, emphysema, hypercapnea, hyperinflation, hyperoxia-induced inflammations, hypoxemia, hypoxia, lung ischemia/reperfusion injury, surgerical lung volume reduction, pulmonary fibrosis, pulmonary hypertension, right ventricular hypertrophy, peritonitis associated with continuous ambulatory peritoneal dialysis, granulocytic ehrlichiosis, sarcoidosis, small airway disease, ventilation-perfusion mismatching, wheeze, colds, gout, alcoholic liver disease, lupus, burn therapy, periodontitis, pre-term labor, cough, pruritis, multi-organ dysfunction, trauma, sprains, contusions, undesired hematopoietic stem cell release, angiogenic ocular disease, ocular inflammation, retinopathy or prematurity, diabetic retinopathy, macular degeneration, corneal neovasularization, tumor angiogenesis, cancer, ormetastasis.

Certain embodiments provided herein describe a method of treating a hyperproliferative condition in a subject in need thereof, comprising administering to the subject a therapeutically effective amount of a combination of: (i) Colchicine, or a pharmaceutically acceptable salt thereof, and (ii) a CXCR-2 inhibitor, or a pharmaceutically acceptable salt thereof. In certain specific embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide or a pharmaceutically acceptable salt thereof. In some embodiments, the hyperproliferative condition is cancer. In certain embodiments, the cancer is multiple myeloma, leukemia, acute lymphocytic leukemia (ALL), acute nonlymphocytic leukemia (ANLL), chronic lymphocytic leukemia (CLL), chronic myelogenous leukemia (CML), lymphoma, Hodgkin's lymphoma, non-Hodgkin's lymphoma, hematologic cancer, nonhematologic cancer, multiple myeloma, brain cancer, cancers of the head and neck, lung cancer, breast cancer, cancers of the reproductive system, prostate cancer, cancers of the digestive system, colorectal cancer, pancreatic cancer, bladder cancer, renal cell carcinoma, cancers of oral cavity, cancer of the tongue, cancer of the mouth, cancer of the pharynx, cancers of the eye and orbit, cancers of the respiratory system, cancers of bones and joints, cancers of soft tissue, skin cancers, cancers of the genital system, cancers of the nervous system, cancers of the lymphatic system, cancers of the endocrine system, esophageal cancer, stomach cancer, cancer of the small intestine, cancers of the urinary system, colon cancer, rectal cancer, anal cancer, anorectal cancer, liver cancer, gallbladder cancer, pancreatic cancer, laryngeal cancer, bronchial cancer, heart cancer, melanoma, basal cell carcinoma, squamous cell carcinoma, uterine cancer, cervical cancer, ovarian cancer, vulvar cancer, vaginal cancer, prostate cancer, testicular cancer, penile cancer, urinary bladder cancer, cancer of the kidney, renal cancer, pelvic cancer, urethral cancer, thyroid cancer, chronic lymphocytic leukemia, cutaneous T-cell lymphoma, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, or Wilm's tumor, or any combination thereof.

Also provided herein in some embodiments is a pharmaceutical composition, comprising a pharmaceutically acceptable excipient and a combination of: (i) Colchicine, or a pharmaceutically acceptable salt thereof, and (ii) a CXCR-2 inhibitor, or a pharmaceutically acceptable salt thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B depict results of a cell migration assay with neutrophil and PBMC counts for compounds 1 and 2 (see Example 2). FIG. 1A depicts the neutrophil counts. FIG. 1B depicts the PBMC counts.

FIGS. 2A-2C depict the results from the rat air pouch model of crystal-induced arthropathy with compound 2 (see Example 4). FIG. 2A depicts the average exudate volume. FIG. 2B depicts the total white blood cell counts. FIG. 2C depicts the neutrophil counts.

FIGS. 3A-3C depict the results from the rat air pouch model of crystal-induced arthropathy with compound 2 in combination with colchicine (see Example 5). FIG. 3A depicts the average exudate volume. FIG. 3B depicts the total white blood cell counts. FIG. 3C depicts the neutrophil counts.

FIGS. 4A-4C depict the results from the rat air pouch model of crystal-induced arthropathy with compounds 2, 3, and 4 (see Example 6). FIG. 4A depicts the average exudate volume. FIG. 4B depicts the total white blood cell counts. FIG. 4C depicts the neutrophil counts.

FIGS. 5A-5C depict results from the rat air pouch model of crystal-induced arthropathy with compound 3 and compound 4 in combination with colchicine (see Example 7). FIG. 5A depicts the average exudate volume. FIG. 5B depicts the total white blood cell counts. FIG. 5C depicts the neutrophil counts.

FIGS. 6A-6C depict results from the rat air pouch model of crystal-induced arthropathy with compound 3 in combination with colchicine (see Example 8). FIG. 6A depicts the average exudate volume. FIG. 6B depicts the total white blood cell counts. FIG. 6C depicts the neutrophil counts.

FIGS. 7A-7C depict results from the rat air pouch model of crystal-induced arthropathy with compound 4 in combination with colchicine (see Example 9). FIG. 7A depicts the average exudate volume. FIG. 7B depicts the total white blood cell counts. FIG. 7C depicts the neutrophil counts.

FIGS. 8A-8C depict results from the rat air pouch model of crystal-induced arthropathy with compounds 3 and 4 in combination with colchicine (see Example 10). FIG. 8A depicts the average exudate volume. FIG. 8B depicts the total white blood cell counts. FIG. 8C depicts the neutrophil counts.

FIGS. 9A-9D depict amounts of exudate for IL-1β and MPO content in the presence of colchicine, compound 4, and the colchicine/compound 4 combination of groups 1, 2, 3, 6, and 10 from example 9 and groups 1, 2, 3, 6, and 9 from example 10. FIG. 9A depicts amounts of IL-1β for example 10. FIG. 9B depicts amounts of IL-1β for example 9. FIG. 9C depicts amounts of MPO for example 10. FIG. 9D depicts amounts of MPO for example 9.

FIGS. 10A-10D depict differences from positive control (A) of exudate in the presence of colchicine, compound 4, and the colchicine/compound 4 combination for IL-1β and MPO of groups 1, 2, 3, 6, and 10 from example 9 and groups 1, 2, 3, 6, and 9 from example 10. FIG. 10A depicts A from positive control of IL-1β for example 10. FIG. 10B depicts A from positive control of IL-1β for example 9. FIG. 10C depicts A from positive control of MPO for example 10. FIG. 10D depicts A from positive control of MPO for example 9.

### DETAILED DESCRIPTION OF THE INVENTION

### Definitions

The term “subject”, as used herein in reference to individuals suffering from a disorder, and the like, encompasses mammals and non-mammals. In one embodiment of the methods and compositions provided herein, the mammal is a human.

The terms “effective amount”, “therapeutically effective amount” or “pharmaceutically effective amount” as used herein, refer to an amount of at least one agent or compound being administered that is sufficient to treat or prevent the particular disease or condition. The result is the reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case is determined using techniques such as a dose escalation study.

A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

A “synergistically effective” therapeutic amount of an agent or therapy is an amount that, when combined with an effective or sub-therapeutic amount of another agent or therapy, produces a greater effect than the expected additive effects of each of the two agents or therapies. The term “greater effect” encompasses not only a reduction in symptoms of the disorder to be treated, but also an improved side effect profile, improved tolerability, improved patient compliance, improved efficacy, or any other improved clinical outcome.

The terms “synergistic” and “synergistically” as applied to the effect of two or more pharmaceutically active ingredients used in combination (whether simultaneously or sequentially) refer to a greater effect than the expected additive effects of the two agents.

The term “about” refers to +10% of a stated number or value.

In the context of the present specification, the term “therapy” also includes “prophylaxis” unless there are specific indications to the contrary. The terms “therapeutic” and “therapeutically” should be construed accordingly.

### Agents

Colchicine

Colchicine is used to treat acute gout flares (and the symptoms associated therewith) as well as for prophylaxis of acute gout flares. While colchicine is neither an analgesic nor a uricosuric and will not prevent progression to chronic gouty arthritis, it does have a prophylactic, suppressive effect that helps to reduce the incidence of acute attacks and relieve residual pain.

Colchicine is rapidly absorbed from the gastrointestinal tract. Peak concentrations occur in 0.5 to 2 hours. The drug and its metabolites are distributed in leukocytes, kidneys, liver, spleen and the intestinal tract. Colchicine is metabolized in the liver and excreted primarily in the feces with 10-20% eliminated unchanged in the urine.

COLCRYS (colchicine, USP) is indicated for both the prophylaxis and treatment of gout flares (see for example the COLCRYS prescribing information or U.S. Pat. Nos. 7,964,647 and 7,981,938). Prescribing information of COLCRYS requires:

0.6 mg once or twice daily to a maximum dose of 1.2 mg/day for prophylaxis of gout flares; and
1.2 mg at the first sign of a gout flare followed by 0.6 mg one hour later for treatment of gout flares.

Common side effects from taking COLCYS include diarrhea, nausea, vomiting, abdominal pain and pharyngolaryngeal pain. Warnings regarding the use of COLCRYS include blood dyscrasias (myelosuppression, leukopenia, granulocytopenia, thrombocytopenia and aplastic anemia); drug interaction with P-gp and/or CYP3A4 inhibitors (resulting in life-threatening interactions and death) and neuromuscular toxicity (myotoxicity including rhabdomyolysis).

The most frequently reported adverse side effects to colchicine therapy are gastrointestinal, specifically diarrhea; abdominal pain with cramps; nausea; and vomiting. Less frequently or rarely reported adverse side effects associated with colchicine therapy include anorexia, agranulocytosis, allergic dermatitis, allergic reactions, alopecia, angioedema, aplastic anemia, bone marrow depression, myopathy, neuropathy, skin rash, thrombocytopenic disorder and urticaria.

Clinical trial studies on colchicine (see U.S. Pat. No. 7,964,647) showed that a “standard” dose regimen of colchicine (1.2 mg administered at the onset of an acute gout attack followed by 0.6 mg every hour thereafter for 6 hours) resulted in more gastrointestinal side effects than placebo (73% vs. 19%) and more diarrhea than placebo (73% vs. 14%). Severe diarrhea occurred in 19% of patients and vomiting occurred in 15% of patients. A “lower” dose regimen (1.2 mg colchicine administered at the onset of an acute gout attack followed by 0.6 mg after one hour) resulted in more gastrointestinal side effects than placebo (24% vs. 19%) and more diarrhea than placebo (22% vs. 14%). The “standard” dose of colchicine used to treat or prevent an attack of acute gouty arthritis was 1.0-1.2 mg, typically followed by 0.5-0.6 mg every hour, until pain is relieved or until diarrhea ensues (“diarrheal dose”). The dosing should be stopped if there is gastrointestinal discomfort or diarrhea. (Opiates may be needed to control diarrhea.) In subsequent attacks, the patient should be able to judge medication requirement accurately enough to stop short of the diarrheal dose. The total amount of colchicine needed to control pain and inflammation during an attack was believed to be in the 4-8 mg range. An interval of three days between colchicine courses was advised in order to minimize the possibility of cumulative toxicity.

CXCR-2 Inhibitors

Chemokines play an important role in immune and inflammatory responses in various diseases and disorders. These small secreted molecules are a growing superfamily of 8-14 kDa proteins characterized by a conserved cysteine motif.

The chemokine superfamily comprises three groups exhibiting characteristic structural motifs, the C—X—C, C—C and C—X₃—C families. The C—X—C chemokines include several potent chemo-attractants and activators of neutrophils such as interleukin-8 (IL-8) and neutrophil-activating peptide 2 (NAP-2).

Studies have demonstrated that the actions of the chemokines are mediated by subfamilies of G protein-coupled receptors, among which are the receptors designated CXCR-1, CXCR-2, CXCR-3, CXCR-4 and CXCR-5 (for the C—X—C family). Only IL-8, and certain other C—X—C chemokines that bind IL-8 receptors, are known to chemo-attract human neutrophils. Of the human C—X—C chemokine receptors identified to date (CXCR-1, 2, 3, 4 and 5), only CXCR-1 and CXCR-2 act as high-affinity IL-8 receptors. C—X—C chemokines that chemo-attract neutrophils share specific sequence motifs. These receptors represent good targets for drug development since agents that modulate these receptors would be useful in the treatment of immune and inflammatory related disorders and diseases.

CXCR-2 is an I-L8 receptor. Chemokines that bind CXCR-2 are required for neutrophilic inflammation in acute gout (Terkaltaub et al, Arthritis & Rheumatism, (1988), Vol 41, (No 5) pp 900-909). Urate crystals can initiate, amplify and sustain an intense inflammatory attack because they stimulate the synthesis and release of humoral and cellular inflammatory mediators. Neutrophilic synovitis is the hallmark of an acute gouty attack. Neutrophils are rare in normal synovial fluid. Monosodium urate monohydrate (MSUM) crystals from supersaturated extracellular fluids are deposited in synovial tissue, which activates resident mononuclear phagocytes and synovial lining cells to release neutrophil chemotaxins—IL-8 and closely related, neutrophil chemotactic C—X—C chemokines. The newly generated neutrophil chemotaxins direct neutrophil transmigration. MSUM crystals interact with the phagocyte through two broad mechanisms. First, the crystals activate cells as opsonized and phagocytosed particles, eliciting the phagocyte response and release of inflammatory mediators. Second, urate crystals interact directly with lipid membranes and proteins, leading to the activation of several signal transduction pathways. These steps are critical for crystal-induced interleukin (IL)-8 expression. IL-8 is abundant in the synovial fluid in both acute gout and pseudogout. The rapid release of IL-8 (and other neutrophil chemotactic C—X—C chemokines) by crystal-activated resident mononuclear phagocytes and synovial lining cells triggers acute gout. Once in the synovial tissue, the neutrophils follow concentration gradients of chemoattractants such as C5a, leukotriene B4, platelet-activating factor, IL-1, and IL-8. Of these factors, IL-8 plays a central role in neutrophil invasion, accounting for approximately 90% of the neutrophil chemotactic activity of monocytes in response to urate crystals.

It has been postulated that colchicine's ability to suppress certain neutrophil responses to IL-8 could contribute to its' preventive and therapeutic properties in acute gout. Neutralization of IL-8 or its receptor CXCR-2 may substantially reduce the IL-8-induced neutrophilic inflammatory process and provide a potential therapeutic target in gout.

In some instances, activation of keratinocytes mediated by CXCR2 contributes to the characteristic epidermal changes observed in dermatological diseases (e.g., psoriasis). Increased expression of CXCR-2 has been observed in psoriatic epidermis and suggested to contribute to psoriatic hyperproliferation. (J. Invest. Dermatol. 1998; 110: 90-94). Some embodiments provided herein describe a CXCR-2 inhibitor, alone or in combination with a second agent (e.g., colchicine), for the treatment of a dermatological disease or disorder.

In some embodiments, the following compounds provided in the following table, or pharmaceutically acceptable salts thereof, may be useful to treat diseases in which the chemokine receptor belongs to the CXC chemokine receptor subfamily, more conveniently the target chemokine receptor is the CXCR-2 receptor. In some embodiments, the compounds 1, 2, 3, and 4 are CXCR-2 inhibitors.

| | | | |
|---|---|---|---|
| 1 | 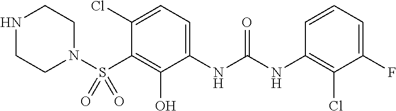 [SMILES: O=C(Nc1ccc(Cl)c(S(=O)(=O)N2CCNCC2)c1O)Nc1cccc(F)c1Cl] | 1-(4-chloro-2-hydroxy-3-(piperazin-1- ylsulfonyl)phenyl)-3-(2-chloro-3- fluorophenyl)urea | 463.3 |
| 2 | 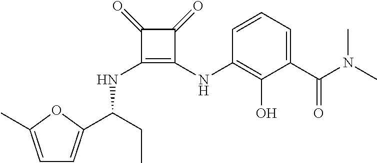 | (R)-2-hydroxy-N,N-dimethyl-3-((2-((1-(5- methylfuran-2-yl)propyl)amino)-3,4- dioxocyclobut-1-en-1-yl)amino)benzamide | 397.4 |
| 3 | 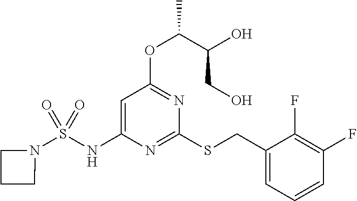 [SMILES: C[C@@H](Oc1cc(NS(=O)(=O)N2CCC2)nc(SCc2cccc(F)c2F)n1)[C@@H](O)CO] | N-(2-((2,3-difluorobenzyl)thio)-6-(((2R,3S)- 3,4-dihydroxybutan-2-yl)oxy)pyrimidin-4- yl)azetidine-1-sulfonamide | 476.5 |
| 4 | 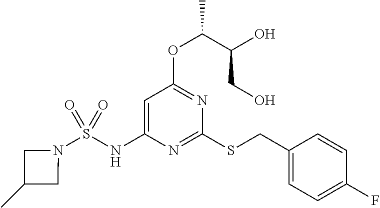 [SMILES: CC1CN(S(=O)(=O)Nc2cc(O[C@H](C)[C@@H](O)CO)nc(SCc3ccc(F)cc3)n2)C1] | N-(6-(((2R,3S)-3,4-dihydroxy butan-2-yl)oxy)- 2-((4-fluoro benzyl)thio)pyrimidin-4-yl)-3- methylazetidine-1-sulfonamide | 472.6 |

In some embodiments, compounds 1, 2, 3, and 4, or pharmaceutically acceptable salts thereof, are useful as pharmaceuticals, in particular as modulators of chemokine receptor (especially CXCR-2) activity, and may be used in the treatment (therapeutic or prophylactic) of conditions/diseases in human and non-human animals which are exacerbated or caused by excessive or unregulated production of chemokines. In particular embodiments, the condition/disease is gout flare. Thus, the present invention provides compounds 1, 2, 3 and 4, or pharmaceutically acceptable salts thereof, for use in therapy. In some embodiments, the present invention provides compounds 4, or pharmaceutically acceptable salts thereof, for use in therapy.

Compound 3 (N-(2-((2,3-difluorobenzyl)thio)-6-(((2R,3S)-3,4-dihydroxybutan-2-yl)oxy)pyrimidin-4-yl)azetidine-1-sulfonamide) and compound 4 (N-(6-(((2R,3S)-3,4-dihydroxy butan-2-yl)oxy)-2-((4-fluoro benzyl)thio)pyrimidin-4-yl)-3-methylazetidine-1-sulfonamide) are pyrimidinyl sulphonamides and are useful as modulators of chemokine receptors.

WO 2004/011443 describes pyrimidinyl sulphonamide derivatives for use as modulators of chemokine receptors.

The in vitro potency and PK parameters of compound 3 are described in WO 2006/024823 and WO 2010/007427. Compound 3 displays good bioavailability in rat (49%), a long half-life in dog, good solubility properties and high potency. Compound 3 was in Phase II trials for COPD. The preparation of compound 3, along with six crystalline forms, is described in WO 2012/007748.

The preparation of compound 4, along with several distinct crystalline forms, is described in WO 2013/008002.

Examples of other CXCR-2 inhibitors include but are not limited to, AZD-8309, AZ-10397767, elubrixin, danirixin, navarixin, reparixin, ladarixin, and meraxin. Additional examples of other CXCR-2 inhibitors include, but are not limited to, the compounds in the following table:

| | |
|---|---|
| 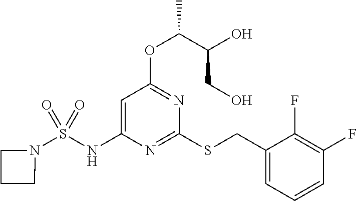 [SMILES: C[C@@H](Oc1cc(NS(=O)(=O)N2CCC2)nc(SCc2cccc(F)c2F)n1)[C@@H](O)CO] | N-(2-(2,3-difluorobenzylthio)-6- ((2R,3R)-3,4-dihydroxybutan-2- ylamino)pyrimidin-4-yl)azetidine- 1-sulfonamide |
| 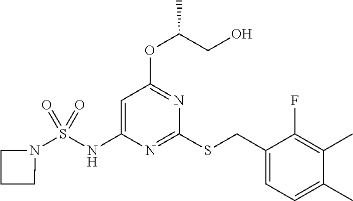 [SMILES: Cc1ccc(CSc2nc(NS(=O)(=O)N3CCC3)cc(O[C@H](C)CO)n2)c(F)c1C] | (R)-N-(2-(2-fluoro-3,4- dimethylbenzylthio)-6-(1- hydroxypropan-2- ylamino)pyrimidin-4-yl)azetidine- 1-sulfonamide |
| 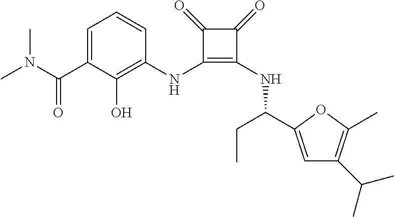 [SMILES: CC[C@H](Nc1c(Nc2cccc(C(=O)N(C)C)c2O)c(=O)c1=O)c1cc(C(C)C)c(C)o1] | (S)-2-hydroxy-3-(2-(1-(4- isopropyl-5-methylfuran-2- yl)propylamino)-3,4- dioxocyclobut-1-enylamino)-N,N- dimethylbenzamide |
| 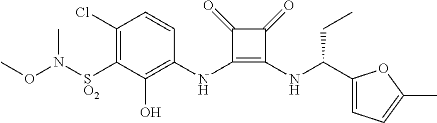 | (R)-6-chloro-2-hydroxy-N- methoxy-N-methyl-3-(2-(1-(5- methylfuran-2-yl)propylamino)- 3,4-dioxocyclobut-1- enylamino)benzenesulfonamide |
| 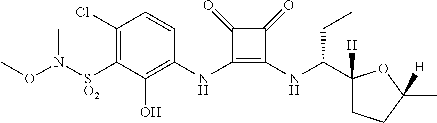 | 6-chloro-2-hydroxy-N-methoxy- N-methyl-3-(2-((R)-1-((2R,5R)-5- methyltetrahydrofuran-2- yl)propylamino)-3,4- dioxocyclobut-1- enylamino)benzenesulfonamide |
| 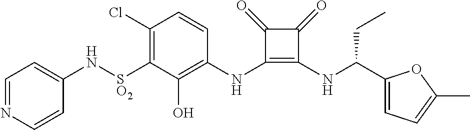 [SMILES: CC[C@@H](Nc1c(Nc2ccc(Cl)c(S(=O)(=O)Nc3ccncc3)c2O)c(=O)c1=O)c1ccc(C)o1] | (R)-6-chloro-2-hydroxy-3-(2-(1- (5-methylfuran-2- yl)propylamino)-3,4- dioxocyclobut-1-enylamino)-N- (pyridin-4-yl)benzenesulfonamide |
| 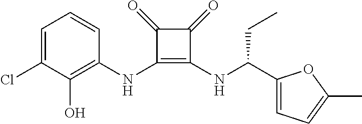 [SMILES: CC[C@@H](Nc1c(Nc2cccc(Cl)c2O)c(=O)c1=O)c1ccc(C)o1] | (R)-3-(3-chloro-2- hydroxyphenylamino)-4-(1-(5- methylfuran-2- yl)propylamino)cyclobut-3-ene- 1,2-dione |
| 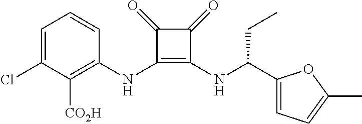 [SMILES: CC[C@@H](Nc1c(Nc2cccc(Cl)c2C(=O)O)c(=O)c1=O)c1ccc(C)o1] | (R)-2-chloro-6-(2-(1-(5- methylfuran-2-yl)propylamino)- 3,4-dioxocyclobut-1- enylamino)benzoic acid |
| 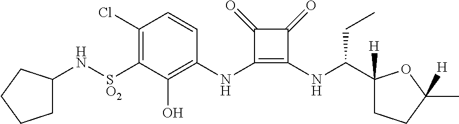 [SMILES: CC[C@@H](Nc1c(Nc2ccc(Cl)c(S(=O)(=O)NC3CCCC3)c2O)c(=O)c1=O)[C@H]1CC[C@@H](C)O1] | 6-chloro-N-cyclopentyl-2- hydroxy-3-(2-((R)-1-((2R,5R)-5- methyltetrahydrofuran-2- yl)propylamino)-3,4- dioxocyclobut-1- enylamino)benzenesulfonamide |
| 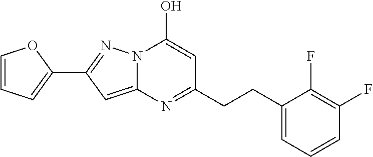 [SMILES: Oc1cc(CCc2cccc(F)c2F)nc2cc(-c3ccco3)nn12] | 5-(2,3-difluorophenethyl)-2- (furan-2-yl)pyrazolo[1,5- a]pyrimidin-7-ol |
| 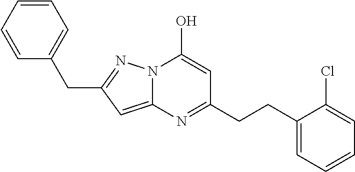 [SMILES: Oc1cc(CCc2ccccc2Cl)nc2cc(Cc3ccccc3)nn12] | 2-benzyl-5-(2- chlorophenethyl)pyrazolo[1,5- a]pyrimidin-7-ol |
| 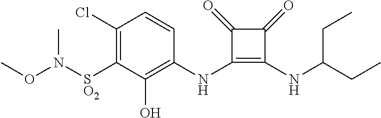 [SMILES: CCC(CC)Nc1c(Nc2ccc(Cl)c(S(=O)(=O)N(C)OC)c2O)c(=O)c1=O] | 6-chloro-3-(3,4-dioxo-2-(pentan- 3-ylamino)cyclobut-1- enylamino)-2-hydroxy-N- methoxy-N- methylbenzenesulfonamide |
| 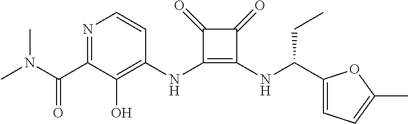 | (R)-3-hydroxy-N,N-dimethyl-4- (2-(1-(5-methylfuran-2- yl)propylamino)-3,4- dioxocyclobut-1- enylamino)picolinamide |
| 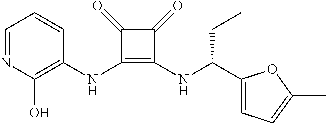 [SMILES: CC[C@@H](Nc1c(Nc2cccnc2O)c(=O)c1=O)c1ccc(C)o1] | (R)-3-(2-hydroxypyridin-3- ylamino)-4-(1-(5-methylfuran-2- yl)propylamino)cyclobut-3-ene- 1,2-dione |
| 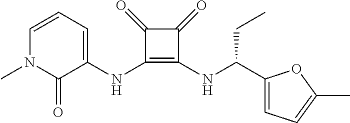 | (R)-3-(1-methyl-2-oxo-1,2- dihydropyridin-3-ylamino)-4-(1- (5-methylfuran-2- yl)propylamino)cyclobut-3-ene- 1,2-dione |
| 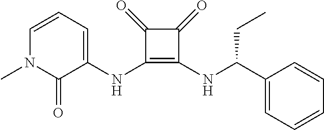 [SMILES: CC[C@@H](Nc1c(Nc2cccn(C)c2=O)c(=O)c1=O)c1ccccc1] | (R)-3-(1-methyl-2-oxo-1,2- dihydropyridin-3-ylamino)-4-(1- phenylpropylamino)cyclobut-3- ene-1,2-dione |
| 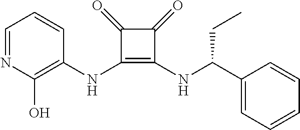 [SMILES: CC[C@@H](Nc1c(Nc2cccnc2O)c(=O)c1=O)c1ccccc1] | (R)-3-(2-hydroxypyridin-3- ylamino)-4-(1- phenylpropylamino)cyclobut-3- ene-1,2-dione |
| 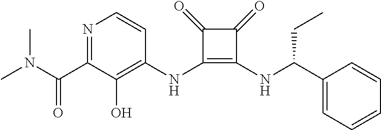 [SMILES: CC[C@@H](Nc1c(Nc2ccnc(C(=O)N(C)C)c2O)c(=O)c1=O)c1ccccc1] | (R)-4-(3,4-dioxo-2-(1- phenylpropylamino)cyclobut-1- enylamino)-3-hydroxy-N,N- dimethylpicolinamide |
| 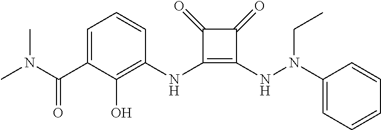 [SMILES: CCN(Nc1c(Nc2cccc(C(=O)N(C)C)c2O)c(=O)c1=O)c1ccccc1] | 3-(2-(2-ethyl-2- phenylhydrazinyl)-3,4- dioxocyclobut-1-enylamino)-2- hydroxy-N,N-dimethylbenzamide |
| 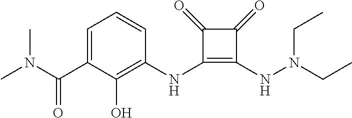 [SMILES: CCN(CC)Nc1c(Nc2cccc(C(=O)N(C)C)c2O)c(=O)c1=O] | 3-(2-(2,2-diethylhydrazinyl)-3,4- dioxocyclobut-1-enylamino)-2- hydroxy-N,N-dimethylbenzamide |
| 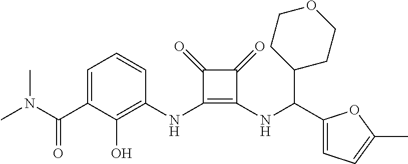 | 2-hydroxy-N,N-dimethyl-3-(2- ((5-methylfuran-2-yl)(tetrahydro- 2H-pyran-4-yl)methylamino)-3,4- dioxocyclobut-1- enylamino)benzamide |
| 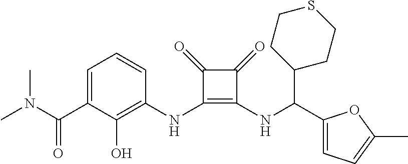 | 2-hydroxy-N,N-dimethyl-3-(2- ((5-methylfuran-2-yl)(tetrahydro- 2H-thiopyran-4-yl)methylamino)- 3,4-dioxocyclobut-1- enylamino)benzamide |
| 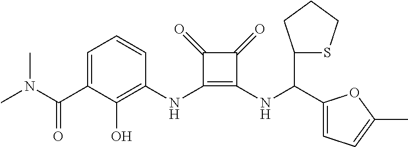 | 2-hydroxy-N,N-dimethyl-3-(2- ((5-methylfuran-2- yl)(tetrahydrothiophen-2- yl)methylamino)-3,4- dioxocyclobut-1- enylamino)benzamide |
| 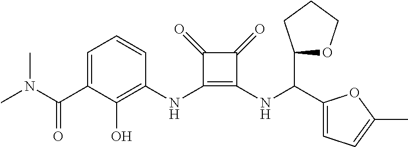 | 2-hydroxy-N,N-dimethyl-3-(2- ((5-methylfuran-2-yl)((R)- tetrahydrofuran-2- yl)methylamino)-3,4- dioxocyclobut-1- enylamino)benzamide |
| 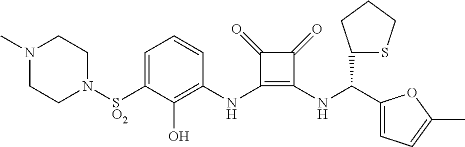 | 3-(2-hydroxy-3-(4- methylpiperazin-1- ylsulfonyl)phenylamino)-4-((S)- (5-methylfuran-2-yl)((R)- tetrahydrothiophen-2- yl)methylamino)cyclobut-3-ene- 1,2-dione |
| 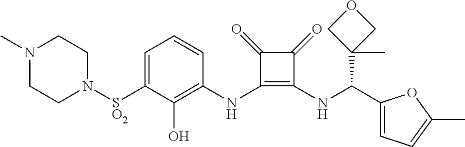 | (R)-3-(2-hydroxy-3-(4- methylpiperazin-1- ylsulfonyl)phenylamino)-4-((5- methylfuran-2-yl)(3- methyloxetan-3- yl)methylamino)cyclobut-3-ene- 1,2-dione |
| 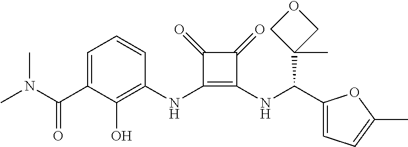 | (R)-2-hydroxy-N,N-dimethyl-3- (2-((5-methylfuran-2-yl)(3- methyloxetan-3-yl)methylamino)- 3,4-dioxocyclobut-1- enylamino)benzamide |
| 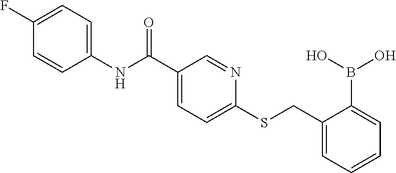 [SMILES: O=C(Nc1ccc(F)cc1)c1ccc(SCc2ccccc2B(O)O)nc1] | 2-((5-(4- fluorophenylcarbamoyl)pyridin-2- ylthio)methyl)phenylboronic acid |
| 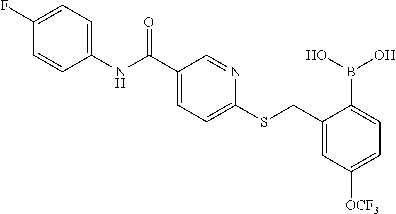 [SMILES: O=C(Nc1ccc(F)cc1)c1ccc(SCc2cc(OC(F)(F)F)ccc2B(O)O)nc1] | 2-((5-(4- fluorophenylcarbamoyl)pyridin-2- ylthio)methyl)-4- (trifluoromethoxy)phenylboronic acid |
| 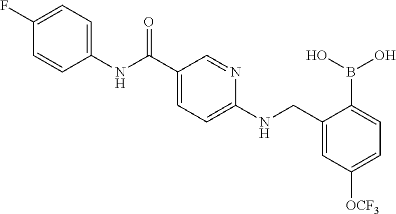 [SMILES: O=C(Nc1ccc(F)cc1)c1ccc(NCc2cc(OC(F)(F)F)ccc2B(O)O)nc1] | 2-((5-(4- fluorophenylcarbamoyl)pyridin-2- ylamino)methyl)-4- (trifluoromethoxy)phenylboronic acid |
| 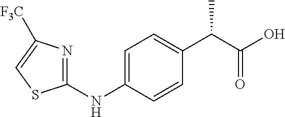 [SMILES: C[C@H](C(=O)O)c1ccc(Nc2nc(C(F)(F)F)cs2)cc1] | (S)-2-(4-(4- (trifluoromethyl)thiazol-2- ylamino)phenyl)propanoic acid |
| 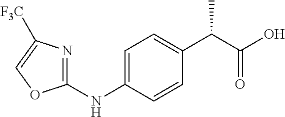 [SMILES: C[C@H](C(=O)O)c1ccc(Nc2nc(C(F)(F)F)co2)cc1] | (S)-2-(4-(4- (trifluoromethyl)oxazol-2- ylamino)phenyl)propanoic acid |
| 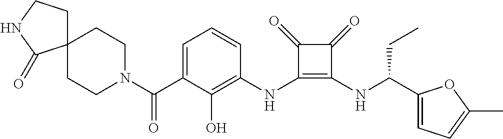 [SMILES: CC[C@@H](Nc1c(Nc2cccc(C(=O)N3CCC4(CCNC4=O)CC3)c2O)c(=O)c1=O)c1ccc(C)o1] | (R)-3-(2-hydroxy-3-(1-oxo-2,8- diazaspiro[4.5]decane-8- carbonyl)phenylamino)-4-(1-(5- methylfuran-2- yl)propylamino)cyclobut-3-ene- 1,2-dione |
| 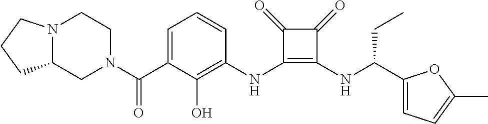 [SMILES: CC[C@@H](Nc1c(Nc2cccc(C(=O)N3CCN4CCC[C@H]4C3)c2O)c(=O)c1=O)c1ccc(C)o1] | 3-(2-hydroxy-3-((S)- octahydropyrrolo[1,2-a]pyrazine- 2-carbonyl)phenylamino)-4-((R)- 1-(5-methylfuran-2- yl)propylamino)cyclobut-3-ene- 1,2-dione |
| 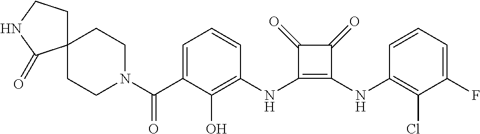 [SMILES: O=C(c1cccc(Nc2c(Nc3cccc(F)c3Cl)c(=O)c2=O)c1O)N1CCC2(CCNC2=O)CC1] | 3-(2-chloro-3- fluorophenylamino)-4-(2- hydroxy-3-(1-oxo-2,8- diazaspiro[4.5]decane-8- carbonyl)phenylamino)cyclobut- 3-ene-1,2-dione |
| 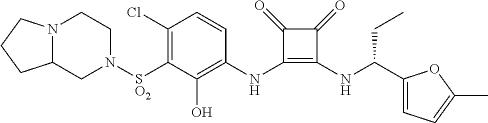 [SMILES: CC[C@@H](Nc1c(Nc2ccc(Cl)c(S(=O)(=O)N3CCN4CCCC4C3)c2O)c(=O)c1=O)c1ccc(C)o1] | 3-(4-chloro-3- (hexahydropyrrolo[1,2-a]pyrazin- 2(1H)-ylsulfonyl)-2- hydroxyphenylamino)-4-((R)-1- (5-methylfuran-2- yl)propylamino)cyclobut-3-ene- 1,2-dione |
| 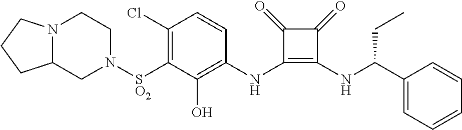 [SMILES: CC[C@@H](Nc1c(Nc2ccc(Cl)c(S(=O)(=O)N3CCN4CCCC4C3)c2O)c(=O)c1=O)c1ccccc1] | 3-(4-chloro-3- (hexahydropyrrolo[1,2-a]pyrazin- 2(1H)-ylsulfonyl)-2- hydroxyphenylamino)-4-((R)-1- phenylpropylamino)cyclobut-3- ene-1,2-dione |
| 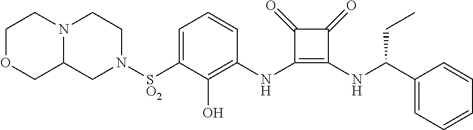 [SMILES: CC[C@@H](Nc1c(Nc2cccc(S(=O)(=O)N3CCN4CCOCC4C3)c2O)c(=O)c1=O)c1ccccc1] | 3-(3-(hexahydropyrazino[2,1- c][1,4]oxazin-8(1H)-ylsulfonyl)- 2-hydroxyphenylamino)-4-((R)-1- phenylpropylamino)cyclobut-3- ene-1,2-dione |
| 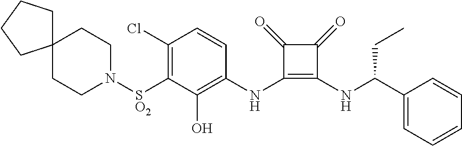 [SMILES: CC[C@@H](Nc1c(Nc2ccc(Cl)c(S(=O)(=O)N3CCC4(CCCC4)CC3)c2O)c(=O)c1=O)c1ccccc1] | 3-(3-(8-azaspiro[4.5]decan-8- ylsulfonyl)-4-chloro-2- hydroxyphenylamino)-4-((R)-1- phenylpropylamino)cyclobut-3- ene-1,2-dione |
| 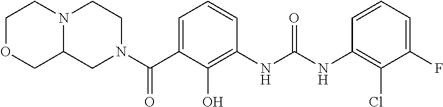 [SMILES: O=C(Nc1cccc(C(=O)N2CCN3CCOCC3C2)c1O)Nc1cccc(F)c1Cl] | 1-(2-chloro-3-fluorophenyl)-3-(2- hydroxy-3- (octahydropyrazino[2,1- c][1,4]oxazine-8- carbonyl)phenyl)urea |
| 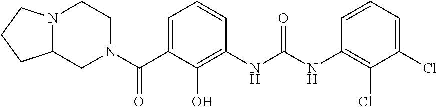 [SMILES: O=C(Nc1cccc(C(=O)N2CCN3CCCC3C2)c1O)Nc1cccc(Cl)c1Cl] | 1-(2,3-dichlorophenyl)-3-(2- hydroxy-3-(octahydropyrrolo[1,2- a]pyrazine-2- carbonyl)phenyl)urea |

In a still further aspect, the present invention provides the use of compounds 1, 2, 3 or 4, or pharmaceutically acceptable salts thereof, as hereinbefore defined, for use as a medicament for the treatment of crystal arthropathy disease, gout, gouty arthritis and gout flare.

In a further aspect, the present invention provides the use of compounds 1, 2, 3 or 4, or pharmaceutically acceptable salts thereof, as hereinbefore defined in the manufacture of a medicament for use in therapy.

In a still further aspect, the present invention provides the use of compounds 1, 2, 3 or 4, or pharmaceutically acceptable salts thereof, as hereinbefore defined in the manufacture of a medicament for the treatment of human diseases or conditions in which modulation of chemokine receptor activity is beneficial.

In a still further aspect, the present invention provides the use of compounds 1, 2, 3 or 4, or pharmaceutically acceptable salts thereof, as hereinbefore defined in the manufacture of a medicament for the treatment of crystal arthropathy disease, gout, gouty arthritis and gout flare.

### Methods

Described herein is a method of treating crystal arthopathy disease, gout, gouty arthritis or gout flare, in a patient suffering from, or at risk of, said disease, which comprises administering to the patient a therapeutically effective amount of a CXCR-2 inhibitor, or a pharmaceutically acceptable salt thereof. In some embodiments, the CXCR-2 inhibitor is compound 1, 2, 3 or 4, or pharmaceutically acceptable salts thereof. In some embodiments, the CXCR-2 inhibitor is compound 3 or 4, or pharmaceutically acceptable salts thereof. In some embodiments, the CXCR-2 inhibitor is compound 3 or pharmaceutically acceptable salts thereof. In some embodiments, the CXCR-2 inhibitor is compound 4 or pharmaceutically acceptable salts thereof.

Crystal Arthropathy Disease

Crystal arthropathy is a class of joint disorder (arthropathy) characterized by accumulation of tiny crystals in one or more joints. Polarizing microscopy allows identification of different microcrystals including monosodium urate, calcium-pyrophosphate (chondrocalcinosis or pseudogout), calcium hydroxyapatite, and calcium oxalate. Risk factors for developing crystal arthropathy include obesity, renal failure, hyperphosphatemia, hyperparathyroidism, hypercalcemia and tissue damage (dystrophic calcification).

Provided herein are methods for treating a crystal arthropathy disease by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods for treating a crystal arthropathy disease comprise administering to a subject in need thereof N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods for treating a crystal arthropathy disease comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof.

Also described herein are methods for treating a crystal arthropathy disease by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof.

In some embodiments, the crystal arthropathy disease is monosodium urate crystal disease, uric acid crystal disease, calcium pyrophosphate disease, calcium crystal disease, basic calcium phosphate hydroxy-apatite deposition disease, calcific periarthritis disease, calcium oxalate aluminium phosphate deposition disease, xanthine deposition disease, Cysteine/cystine deposition disease, Charcot-Leyden disease, or lysophospho-lipase deposition disease. In some embodiments, the crystal arthropathy disease is monosodium urate crystal disease. In some embodiments, the crystal arthropathy disease is uric acid crystal disease. In some embodiments, the crystal arthropathy disease is calcium pyrophosphate disease. In some embodiments, the crystal arthropathy disease is calcium crystal disease. In some embodiments, the crystal arthropathy disease is basic calcium phosphate hydroxy-apatite deposition disease. In some embodiments, the crystal arthropathy disease is calcific periarthritis disease. In some embodiments, the crystal arthropathy disease is calcium oxalate aluminium phosphate deposition disease. In some embodiments, the crystal arthropathy disease is xanthine deposition disease. In some embodiments, the crystal arthropathy disease is Cysteine/cystine deposition disease. In some embodiments, the crystal arthropathy disease is Charcot-Leyden disease. In some embodiments, the crystal arthropathy disease is lysophospho-lipase deposition disease.

Disease Characterized by Accumulation of Crystals

Also described herein are methods for treating a disease characterized by the accumulation of crystals in one or more joints by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods for treating a disease characterized by the accumulation of crystals in one or more joints comprises administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods for treating a disease characterized by the accumulation of crystals in one or more joints comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof.

Also described herein are methods for treating a disease characterized by the accumulation of crystals in one or more joints by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof.

Gout

Gout is a disease caused by buildup of uric acid (due to either an overproduction of uric acid or, more commonly, a reduced ability of the kidney to excrete uric acid) leading to crystal deposition in joints and the surrounding tissues, provoking an inflammatory response. Acute gouty arthritis (or a “gout flare” or a “gout attack”) is a sudden attack of pain, frequently starting during the night, and usually involves only one or a few joints; the big toe, knee, or ankle joints are most often affected. The pain has been described as throbbing, crushing, burning or excruciating. The affected joint may show signs of warmth or hotness, redness, tenderness, swelling and/or stiffness. Low-grade fever may also be present. The crystals inside the joint cause intense pain whenever the affected area is moved. Inflammation of the tissues surrounding the affected joint may cause the skin to swell, and become tender and sore to even the slightest pressure.

Chronic gout involves repeated attacks of joint pain, which often last longer. Several gout attacks within a year, can lead to joint deformity and limited motion in joints. Uric acid deposits, called tophi, develop in cartilage tissue, tendons, and soft tissues, though usually develop only after a patient has suffered from the disease for many years. Deposits also can occur in the kidneys, leading to chronic kidney failure.

Gout Flares

Also described herein are methods for treating a gout flare experienced by a subject by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods for treating a gout flare experienced by a subject comprise administering to a subject in need thereof N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods for treating a gout flare experienced by a subject comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof.

Also described herein are methods for treating a gout flare experienced by a subject by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof.

Also described herein are methods for increasing the rapidity of relief of symptoms in a subject experiencing a gout flare or early symptoms of a gout flare by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods for increasing the rapidity of relief of symptoms in a subject experiencing a gout flare or early symptoms of a gout flare comprise administering to a subject in need thereof N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods for increasing the rapidity of relief of symptoms in a subject experiencing a gout flare or early symptoms of a gout flare comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof.

Also described herein are methods for increasing the rapidity of relief of symptoms in a subject experiencing a gout flare or early symptoms of a gout flare by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof.

Also described herein are methods for reducing the duration or intensity of gout flares experienced by a subject by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods for reducing the duration or intensity of gout flares experienced by a subject comprise administering to a subject in need thereof N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods for reducing the duration or intensity of gout flares experienced by a subject comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof.

Also described herein are methods for reducing the duration or intensity of gout flares experienced by a subject by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof.

Also described herein are methods of preventing or reducing the incidence of a gout flare by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods of preventing or reducing the incidence of a gout flare comprise administering to a subject in need thereof N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods of preventing or reducing the incidence of a gout flare comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof.

Also described herein are methods preventing or reducing the incidence of a gout flare by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof.

In some instances, an increase in gout flares occurs after initiation of gout therapy (e.g., uric acid-lowering therapy) due to changing serum uric acid levels resulting in mobilization of urate from tissue deposits. In some instances, prophylactic therapy is beneficial for the first 6 months of uric acid-lowering therapy. Described herein are methods of preventing or reducing the incidence of a gout flare associated with gout therapy by administering to a subject in need thereof a CXCR-2 inhibitor. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof, or N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof. In some embodiments, the methods of preventing or reducing the incidence of a gout flare associated with gout therapy comprise administering to a subject in need thereof N-(2-((2,3-difluorobenzyl)thio)-6-(((2R,3S)-3,4-dihydroxybutan-2-yl)oxy)pyrimidin-4-yl)azetidine-1-sulfonamide (compound 3), or a pharmaceutically acceptable salt thereof. In some embodiments, the methods of preventing or reducing the incidence of a gout flare associated with gout therapy comprise administering to a subject in need thereof N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof. In some embodiments, the gout therapy comprises treatment with a xanthine oxidase inhibitor, a URAT1 inhibitor, a uricosuric agent, a urate oxidase enzyme, PNP inhibitor, SGLT2 inhibitor or a combination thereof. In some embodiments, the gout therapy is selected from allopurinol, febuxostat, uricase, pegylated uricase, rasburicase, probenecid, sulfinpyrazone, benzbromarone, fenofibrate, lesinurad, zurampic, Verinurad, rhalofenate, oral Bucillamine or combinations thereof.

In some embodiments, the CXCR-2 inhibitor is administered prophylactically for the first 1, 2, 3, 4, 5, 6, 7, 8, 9, 10, 11, or 12 months of uric acid-lowering therapy. In some embodiments, the CXCR-2 inhibitor is administered prophylactically for the first 6 months of uric acid-lowering therapy. In some embodiments, the CXCR-2 inhibitor is administered prophylactically for the first 3-6 months of uric acid-lowering therapy. In some embodiments, the CXCR-2 inhibitor is administered prophylactically for the first 6-9 months of uric acid-lowering therapy. In some embodiments, the CXCR-2 inhibitor is administered prophylactically for the first 9-12 months of uric acid-lowering therapy. In some embodiments, the CXCR-2 inhibitor is administered prophylactically for the first 3-9 months of uric acid-lowering therapy.

Also described herein are methods preventing or reducing the incidence of a gout flare associated with gout therapy by administering to a subject in need thereof 1-(4-chloro-2-hydroxy-3-(piperazin-1-ylsulfonyl)phenyl)-3-(2-chloro-3-fluorophenyl)urea (compound 1), or a pharmaceutically acceptable salt thereof; or (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the gout therapy comprises treatment with a xanthine oxidase inhibitor, a URAT1 inhibitor, a uricosuric agent, a urate oxidase enzyme, PNP inhibitor, SGLT2 inhibitor or a combination thereof. In some embodiments, the gout therapy is selected from allopurinol, febuxostat, uricase, pegylated uricase, rasburicase, probenecid, sulfinpyrazone, benzbromarone, fenofibrate, lesinurad, zurampic, Verinurad, arhalofenate, oral Bucillamine or combinations thereof.

Interleukin-1 (IL-1)- or Myeloperoxidase (MPO)-Mediated Diseases

Some embodiments provided herein describe a method of treating an IL-1- and/or MPO-mediated disease or disorder in a patient in need thereof, the method comprising administering to the patient a therapeutically effective amount of a CXCR-2 inhibitor or a pharmaceutically acceptable salt thereof. Some embodiments provided herein describe a method of treating an IL-1- and/or MPO-mediated disease or disorder in a patient in need thereof, the method comprising administering to the patient a therapeutically effective amount of i) a CXCR-2 inhibitor or a pharmaceutically acceptable salt thereof; and ii) colchicine or a pharmaceutically acceptable salt thereof. Also provided herein in some embodiments is a composition comprising a CXCR2-inhibitor, or pharmaceutically acceptable salt thereof, useful for treating an IL-1- and/or MPO-mediated disease or disorder. Also provided herein in some embodiments are compositions comprising i) a CXCR2-inhibitor or pharmaceutically acceptable salt thereof; and ii) colchicine or a pharmaceutically acceptable salt thereof, wherein the compositions are useful for treating an IL-1- and/or MPO-mediated disease or disorder.

Interleukin-1 (IL-1) consists of two distinct ligands (IL-1α and IL-1β) that signal through the IL-1 type I receptor (IL-1RI). The balance between IL-1 agonists and antagonists plays an essential role in a variety of cardiovascular conditions. For example, following myocardial infarction, IL-1 regulates the inflammatory response and is involved in the development of adverse remodeling by enhancing expression of matrix metalloproteinases. Further, IL-1 signaling may be a mediator in the pathogenesis of heart failure by suppressing cardiac contractility, promoting myocardial hypertrophy and inducing cardiomyocyte apoptosis. A review by Bujak & Frangogiannis (“The role of Interleukin-1 in the pathogenesis of heart disease”, Arch Immunol Ther Exp (Warsz), 2009, 57(3), 165-176) summarizes data showing the significant role of IL-1 signaling in heart disease. IL-1 initiates and propagates inflammation, mainly by inducing a local cytokine network and enhancing inflammatory cell infiltration to affected sites and by augmenting adhesion molecule expression on ECs and leukocytes.

Canakinumab is a selective, high-affinity, inhibitor of IL-1β, a key cytokine in the inflammatory pathway known to drive the continued progression of inflammatory atherosclerosis. Canakinumab works by blocking the action of IL-1β for a sustained period of time, thereby inhibiting inflammation that is caused by its over-production.

A recent Phase III study (CANTOS) involving over 10,000 patients over six years showed canakinumab reduced the risk of major adverse cardiovascular events (MACE), in patients with a prior heart attack and inflammatory atherosclerosis.

The specific targeting of interleukin-1β as a cytokine-based therapy for the secondary prevention of atherosclerotic events rests on several observations. The proinflammatory cytokine interleukin-1β plays multiple roles in the development of atherothrombotic plaque, including the induction of procoagulant activity, the promotion of monocyte and leukocyte adhesion to vascular endothelial cells, and the growth of vascular smooth-muscle cells. In mice, interleukin-1β deficiency reduces lesion formation, whereas in cholesterol-fed pigs, exposure to exogenous interleukin-1β increases intimal medial thickening. The NOD-like receptor protein 3 (NLRP3) inflammasome activates interleukin-1β, a process promoted by cholesterol crystals, neutrophil extracellular traps, tissue hypoxia, and arterial flow patterns that are known to promote focal development of atherosclerosis within arteries. This activation of interleukin-1β stimulates the downstream interleukin-6-receptor signaling pathway, which has been implicated by mendelian randomization studies as a potential causal pathway for atherothrombosis. More recently, studies in parabiotic mice and studies of clonal hematopoiesis have implicated interleukin-1β in processes by which bone marrow activation accelerates atherosclerosis. Furthermore, the expression of specific inflammasome gene modules affecting interleukin-1β has been associated with death from any cause and increased atherosclerosis in elderly patients.

Pro-inflammatory mediators were recently shown to play an important role in tumor-mediated angiogenesis and blocking their function may suppress tumor progression (“The role of IL-1 in tumor-mediated angiogenesis,” Front. Physiol., 2014, 5(114), 1-11).

The IL-1 receptor antagonist (IL-1ra) is a naturally occurring inhibitor to IL-1 and acts by binding to the IL-1 receptor without activating it. The protein IL-1ra has been shown to decrease tumor growth, angiogenesis, and metastases in murine xenograft models. Cancer cells directly produce IL-1 or can induce cells within the tumor microenvironment to do so (“Interleukin-1 and cancer progression: the emerging role of interleukin-1 receptor antagonist as a novel therapeutic agent in cancer treatment,” J Transl Med., 2006, 4:48); studies have documented constitutive IL-1β protein production in human and animal cancer cell lines including sarcomas and ovarian and transitional cell carcinomas (“Biologic basis for interleukin-1 in disease,” Blood. 1996, 87, 2095-2147).

In some instances, high IL-1 concentrations within the tumor microenvironment are associated with a more virulent tumor phenotype. In some embodiments, IL-1 upregulated solid tumors include breast, colon, lung, head and neck cancers, and melanomas. In some instances, patients with IL-1 producing tumors have bad prognoses. Elaraj et al (“The role of interleukin 1 in growth and metastasis of human cancer xenografts. Clin Cancer Res. 2006; 12,1088-1096) evaluated melanoma, non-small cell carcinoma, colon, and squamous cell cancer cell lines for the gene expression of IL-1α and IL-1β via real time quantitative reverse transcriptase PCR and found several of these lines to exhibit significantly increased copy numbers of IL-1α or IL-1β. The expression patterns of IL-1 vary; it is expressed in an autocrine or paracrine fashion. Studies have determined the role of IL-1 in tumor growth, metastasis, and angiogenesis (“IL-1 is required for tumor invasiveness and angiogenesis,” Proc Natl Acad Sci USA, 2003,100, 2645-2650; “Biologic basis for interleukin-1 in disease,” Blood, 1996, 87, 2095-2147).

IL-1 also plays key roles in the pathogenesis of inflammatory skin disorders (e.g., hyperproliferative inflammatory conditions such as psoriasis). For example, increased levels of IL-1 beta have been detected within psoriatic lesions compared with uninvolved skin. Studies have demonstrated that IL-1β is critically involved in the generation of hyperproliferative inflammatory skin alterations. (Clin. Exp. Immunol. 2001; 123: 505-510). Experiments in mice have also revealed that epidermal keratinocytes can secrete large amounts of IL-alpha, which induces an inflammatory response in the skin. (Eur. J. Cell Biol. 2010; 89(9): 638-44.) Polymorphisms in the gene encoding IL-1RA (IL1RN) have been associated with early onset psoriasis, alopecia areata, and cutaneous lupus erythematosus (Immunol Rev. 2008; 223: 20-38.) Further, overexpression of the major epidermal proinflammatory cytokines interleukin (IL) 1 alpha (IL-1α) and 1 beta (IL-1β) is positively correlated with symptom exacerbation and disease progression in psoriasis, atopic dermatitis, neutrophilic dermatoses, skin phototoxicity, and skin cancer. IL-1β and the interleukin-1 receptor I (IL-1RI) have been used as a therapeutic target for some autoinflammatory skin diseases. (Curr. Opin. Investig. Drugs 2010; 11(11): 1211-1220.) In some instances, dysregulation of the IL-1 system leads to the development of dermatological diseases or disorder, such as psoriasis, atopic dermatitis, contact dermatitis and cutaneous lupus erythematosus.

Some embodiments provided herein describe a compound useful for inhibition of neutrophil chemotaxis or neutrophil migration. Chemotaxis plays a critical role in many diverse physiological processes, including the recruitment of leukocytes to sites of infection, trafficking of lymphocytes throughout the human body, and patterning of neuronal cells in the developing nervous system. Because of the essential roles of chemotaxis in development and physiology, improperly guided cell movements, in some instances, cause diverse pathological conditions, including tumor growth, cancer metastasis, inflammation-mediated diseases or disorders, autoimmune diseases or disorders, and chemokine-mediated diseases. In some embodiments, the compounds described herein (e.g., compound 4, alone or in combination with colchicine) are useful for treating tumor growth, cancer, inflammation-mediated diseases or disorders, autoimmune diseases or disorders, and chemokine-mediated diseases.

Myeloperoxidase (MPO) plays a role in the immune response and catalyzes the formation of several reactive oxidant species, which contribute to tissue damage during inflammation. Generation of reactive oxidant species via MPO-catalyzed pathways impacts the inflammatory events that contribute to tissue damage resulting from inflammatory conditions, including atherosclerosis. MPO-catalyzed reactions have been attributed to pro-atherogenic biological activities throughout the evolution of cardiovascular disease, including during initiation, propagation, and acute complication phases of the atherosclerotic process. Components of the MPO pathway represent targets for therapeutic interventions to prevent atherosclerotic cardiovascular disease, (see Nicholls and Hazen, “Myeloperoxidase and Cardiovascular Disease” Arterioscler Thromb Vasc Biol. 2005, 25, 1102-1111).

Elevated levels of MPO have been associated with dermatological disorders. For example, significantly elevated serum levels of MPO in psoriasis patients with and without recognizable atherosclerotic lesions have been observed. Overexpression of MPO in lesional skin by CD11b+ leukocytes has been observed as comparted to psoriatic non-lesional and non-psoriatic skin. (Am. J. Transl. Res. 2014; 6(1): 16-27.)

There were over 7 million heart attacks globally in 2015, with 750,000 in the U.S. and 580,000 in the EU. Despite standard treatment, patients who have had a prior heart attack experience a higher ongoing risk of secondary major adverse cardiovascular events (MACE, a composite of cardiovascular death, non-fatal MI, and non-fatal stroke), wherein the risk is related to the increased inflammation associated with inflammatory atherosclerosis. Indeed, 25% of patients who survive a heart attack experience another event within five years. The recurrent MACE in people with inflammatory atherosclerosis is associated with increased morbidity, mortality, and reduced quality of life and represents a major economic burden on patients and healthcare systems around the world.

Some embodiments provided herein describe compositions and methods for reducing the level of biomarkers in a subject or patient. In some embodiments, the reduced biomarker is Insulin, Myeloperoxidase, Microalbumin, C-reactive protein, Osteopontin, glutathione S transferase α, IL-1β, TNF-α, IL-6, IL-8, SAA, VCAM-1, ICAM-1, NGAL, KIM1, MCP-1, or any combination thereof. In other embodiments, the compositions and methods described herein reduce the levels of myeloperoxidase and IL-1β. In certain embodiments, the compositions and methods described herein reduce the level of IL-1. In certain embodiments, the compositions and methods described herein reduce the level of IL-1β. In other embodiments, the compositions and methods described herein reduce the level of myeloperoxidase.

In some embodiments, the compositions and methods described herein are useful for treating or preventing an interleukin-1 (IL-1)-mediated disease or disorder. In some embodiments, the IL-1-mediated disease or disorder is an inflammation-mediated disease or disorder, an autoimmune disease or disorder, or a hematopoietic disease or disorder. In some embodiments, the IL-1-mediated disease or disorder is a vascular or cardiovascular disease or disorder, a neuroinflammatory disease or disorder, a dermatological disease or skin disorder, pancreatitis, or an inflammatory or allergic disease of the airways. Is some embodiments, the compositions and methods described herein are useful for treating an inflammation-mediated disease or disorder or a neutrophil-mediated disease or disorder.

In some embodiments, the IL-1-mediated disease or disorder is a vascular or cardiovascular disease or disorder. In certain embodiments, the IL-1-mediated disease or disorder is coronary artery disease, coronary heart disease, ischemic heart disease, peripheral arterial disease, cerebrovascular disease, stroke, renal artery stenosis, aortic aneurysm, cardiomyopathy, hypertensive heart disease, high blood pressure, hypertension, heart failure, pulmonary heart disease, cardiac dysrhythmias, abnormalities of heart rhythm, inflammatory heart disease, endocarditis, inflammatory cardiomegaly, myocarditis, valvular heart disease, congenital heart disease, rheumatic heart disease, re-perfusion injury, or atherosclerosis, or any combination thereof.

In some embodiments, the IL-1-mediated disease or disorder is a neuroinflammatory disease or disorder. In some embodiments, the IL-1-mediated disease or disorder is Alzheimer's Disease.

In some embodiments, the IL-1-mediated disease or disorder is pancreatitis. In some embodiments, the IL-1-mediated disease or disorder is acute pancreatitis, chronic pancreatitis, alcohol induced pancreatitis, gallstone induced pancreatitis, drug induced pancreatitis, auto-immune pancreatitis, procedure induced pancreatitis, or trauma induced pancreatitis, or any combination thereof.

In some embodiments, the IL-1-mediated disease or disorder is a dermatological disease or skin disorder. In some embodiments, the dermatological disorder is a hyperproliferative inflammatory skin disease or disorder. In some embodiments, the IL-1-mediated disease or disorder is rosacea, eczema, acne, hidradenitis suppurativa, Palmo-Plantar Pustulosis, Generalized Pustular Psoriasis, Pyoderma Gangrenosum, Erosive Pustular Dermatosis of the Scalp, Sweet's Syndrome, Bowel-associated Dermatosis-arthritis Syndrome, Pustular Psoriasis, Acute Generalized Exanthematous Pustulosis, Keratoderma Blenorrhagicum, Sneddon-Wilkinson Disease, IgA Pemphigus, Amicrobial Pustulosis of the Folds, Infantile Acropustulosis, Transient Neonatal Pustulosis, Neutrophilic Eccrine Hidradenitis, Rheumatoid Neutrophilic Dermatitis, Neutrophilic Urticaria, Dermatitis Herpetiformis, Linear IgA disease (LAD), Inflammatory Epidermolysis Bullosa Aquisita, Alopecia Areata, Autoimmune Angioedema, Autoimmune progesterone dermatitis, Autoimmune urticaria, Bullous pemphigoid, Cicatricial pemphigoid, Dermatitis herpetiformis, Epidermolysis bullosa acquisita, Erythema nodosum, Gestational pemphigoid, Lichen planus, Lichen sclerosus, Morphea, Pemphigus vulgaris, Pityriasis lichenoides et varioliformis acuta, Mucha-Habermann disease, Vitiligo, or Neutrophilic Dermatosis of the Dorsal Hands, or any combination thereof. In certain embodiments, the dermatological disease or skin disorder is rosacea, acne, hidradenitis, suppurative, or a combination thereof. In some embodiments, the dermatological disease or skin disorder is pyoderma gangrenosum. In some embodiments, the dermatological disease or skin disorder is psoriasis, atopic dermatitis, contact dermatitis or cutaneous lupus erythematosus. In some embodiments, the dermatological disease or skin disorder is psoriasis, atopic dermatitis, neutrophilic dermatoses, or skin phototoxicity. In some embodiments, the dermatological disease or skin disorder is psoriasis or atopic dermatitis.

In some embodiments, the IL-1-mediated disease or disorder is an autoimmune disease or disorder. In some embodiments, the IL-1-mediated disease or disorder is Pustular Vasculitis, Small Vessel Vasculitis, Urticarial Vasculitis, Autoimmune urticaria, Medium Vessel Vasculitis, rheumatoid arthritis, Celiac disease, Graves' disease, Sjorgen syndrome, scleroderma, thyroiditis, myasthenia gravis, vasculitis, Addison's disease, autoimmune hepatitis, myocarditis, postmyocardial infarction syndrome, postpericardiotomy syndrome, subacute bacterial endocardititis, Anti-Glomerular Basement Membrane nephritis, Interstitial cystitis, lupus nephritis, systemic lupus, bullous systemic lupus erythmatosus, Primary biliary cirrhosis (PBC), Primary sclerosing cholangitis, Antisynthetase syndrome, Ord's thyroiditis, Autoimmune Oophoritis, Autoimmune orchitis, Autoimmune enteropathy, Chron's disease, microscopic colitis, ulcerative colitis, Antiphospholipid syndrome (APS), Aplastic anemia, Autoimmune hemolytic anemia, Autoimmune lymphoproliferative syndrome, Autoimmune neutropenia, or Autoimmune thrombocytopenic purpura.

In some embodiments, the IL-1-mediated disease or disorder is a hematopoietic disease or disorder. In some embodiments, the IL-1-mediated disease or disorder is an anemia, a blood coagulation disorder, a blood platelet disorder, a blood protein disorder, erythroblastosis, hematologic neoplasm, hemoglobinopathies, a hemorrhagic disorder, a leukocyte disorder, methemoglobinemia, pancytopenia, polycythemia, preleukemia, sulfhemoglobinemia, or thrombophilia.

In some embodiments, the IL-1-mediated disease or disorder is a disease of the respiratory tract, a disease of the bones and/or joints, a skin disease, a disease of the gastrointestinal tract, a disease of central and/or peripheral nervous system, cancer, cystic fibrosis, a burn wound, a chronic skin ulcer, a reproductive disease, a re-perfusion injury, allograft rejection, atherosclerosis, Acquired Immunodeficiency Syndrome (AIDS), lupus erythematosus, systemic lupus erythematosus, Hashimoto's thyroiditis, diabetes mellitus type I, diabetes mellitus type II, nephrotic syndrome, eosinophilia fascitis, hyper IgE syndrome, lepromatous leprosy, and idiopathic thrombocytopenia pupura, post-operative adhesions, sepsis, septic shock, Behcet's Disease, Still's Disease, Erythema Marginatum, Unclassified Periodic Fever Syndromes, Autoinflammatory Syndromes, or Erythema Elevatum Diutinum, or any combination thereof.

In some embodiments, the IL-1-mediated disease or disorder is an inflammatory or allergic disease of the airways. In some embodiments, the IL-1-mediated disease or disorder is chronic bronchitis, chronic obstructive bronchitis (COPD), asthma, bronchiectasis, allergic or non-allergic rhinitis or sinusitis, cystic fibrosis, α-1-antitrypsin deficiency, coughs, pulmonary emphysema, pulmonary fibrosis, idiopathic pulmonary fibrosis, or hyper-reactive airways, or any combination thereof.

In some embodiments, the MPO-mediated disease or disorder is acute myeloid leukemia (AML), chronic myeloid leukemia (CML), polycythemia vera, Hodgkin disease, refractory megaloblastic anemia, aplastic anemia, myelofibrosis with myeloid metaplasia, myelodysplastic syndromes, acute coronary syndrome (ACS), cardiovascular disease, kidney disease, chronic obstructive pulmonary disease (COPD), Alzheimer's disease, inflammatory bowel disease, atherosclerotic disease, or rheumatoid arthritis (RA).

Certain embodiments provided herein describe compositions and methods for treating or preventing a chemokine-mediated disease. In some embodiments, the chemokine mediated disease or disorder is arthritis, chronic obstructive pulmonary disease, adult or acute respiratory distress syndrome, asthma, atherosclerosis, myocardial and renal ischemia/reperfusion injury, peripheral limb ischemia/reperfusion injury, inflammatory bowel disease, ulcerative colitis, Crohn's disease, meconium apriration syndrome, atopic dermatitis, cystic fibrosis, psoriasis, psoriatic arthritis, multiple sclerosis, angiogenesis, restenosis, osteoarthritis, osteoporosis, septic shock, endotoxic shock, gram negative sepsis, toxic shock syndrome, stroke, glomerulonephritis, thrombosis, graft vs. host reaction, allograft rejections, transplant reperfusion injury, early transplantation rejection, acute inflammation, alzheimers disease, malaria, respiratory viruses, herpes viruses, hepatitis viruses, HIV, Kaposi's sarcoma-associated viruses, meningitis, gingivitis, herpes encephalitis, CNS vasculitis, traumatic brain injury, brain ischemia/reperfusion injury, migraine, CNS tumors, subarachnoid hemorrhage, post surgical trauma, interstitial pneumonitis, hypersensitivity, crystal induced arthritis, acute and chronic pancreatitis, hepatic ischemia/reperfusion injury, acute alcoholic hepatitis, necrotizing enterocolitis, chronic sinusitis, uveitis, polymyositis, vasculitis, acne, gastric and duodenal ulcers, intestinal ischemia/reperfusion injury, celiac disease, esophagitis, glossitis, rhinitis, airflow obstruction, airway hyperresponsiveness, bronchiolitis, bronchiolitis obliterans, bronchiolitis obliterans organizing pneumonia, bronchiectasis, chronic bronchitis, cor pulmonae, dyspnea, emphysema, hypercapnea, hyperinflation, hyperoxia-induced inflammations, hypoxemia, hypoxia, lung ischemia/reperfusion injury, surgerical lung volume reduction, pulmonary fibrosis, pulmonary hypertension, right ventricular hypertrophy, peritonitis associated with continuous ambulatory peritoneal dialysis, granulocytic ehrlichiosis, sarcoidosis, small airway disease, ventilation-perfusion mismatching, wheeze, colds, gout, alcoholic liver disease, lupus, burn therapy, periodontitis, pre-term labor, cough, pruritis, multi-organ dysfunction, trauma, sprains, contusions, undesired hematopoietic stem cell release, angiogenic ocular disease, ocular inflammation, retinopathy or prematurity, diabetic retinopathy, macular degeneration, corneal neovasularization, tumor angiogenesis, cancer, ormetastasis.

Also provided herein in some embodiments are compositions and methods for treating a hyperproliferative condition in a subject in need thereof. In some embodiments, the hyperproliferative condition is cancer. In some embodiments, the cancer is multiple myeloma, leukemia, acute lymphocytic leukemia (ALL), acute nonlymphocytic leukemia (ANLL), chronic lymphocytic leukemia (CLL), chronic myelogenous leukemia (CML), lymphoma, Hodgkin's lymphoma, non-Hodgkin's lymphoma, hematologic cancer, nonhematologic cancer, multiple myeloma, brain cancer, cancers of the head and neck, lung cancer, breast cancer, cancers of the reproductive system, prostate cancer, cancers of the digestive system, colorectal cancer, pancreatic cancer, bladder cancer, renal cell carcinoma, cancers of oral cavity, cancer of the tongue, cancer of the mouth, cancer of the pharynx, cancers of the eye and orbit, cancers of the respiratory system, cancers of bones and joints, cancers of soft tissue, skin cancers, cancers of the genital system, cancers of the nervous system, cancers of the lymphatic system, cancers of the endocrine system, esophageal cancer, stomach cancer, cancer of the small intestine, cancers of the urinary system, colon cancer, rectal cancer, anal cancer, anorectal cancer, liver cancer, gallbladder cancer, pancreatic cancer, laryngeal cancer, bronchial cancer, heart cancer, melanoma, basal cell carcinoma, squamous cell carcinoma, uterine cancer, cervical cancer, ovarian cancer, vulvar cancer, vaginal cancer, prostate cancer, testicular cancer, penile cancer, urinary bladder cancer, cancer of the kidney, renal cancer, pelvic cancer, urethral cancer, thyroid cancer, chronic lymphocytic leukemia, cutaneous T-cell lymphoma, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, or Wilm's tumor, or any combination thereof. In some embodiments, the cancer is breast cancer, colon cancer, lung cancer, head and neck cancers, and melanomas. In some embodiments, the compositions and methods described herein suppress or reduce tumor growth. In further or additional embodiments, the tumor is an IL-1 upregulated tumor. In some instances, the tumor is an IL-1 producing tumor.

Combinations

Also described herein are combination therapies wherein any one of the CXCR-2 inhibitors disclosed herein, or pharmaceutically acceptable salts thereof, is administered concurrently or sequentially with an additional therapy and/or an agent for the treatment of crystal arthropathy disease, gout, gouty arthritis or gout flare. In some embodiments, a CXCR-2 inhibitor, or pharmaceutically acceptable salt thereof, is administered concurrently or sequentially with an additional an agent for the treatment of gout, gouty arthritis or gout flare. In some embodiments, the CXCR-2 inhibitor, or pharmaceutically acceptable salts thereof, is administered concurrently or sequentially with colchicine. In some embodiments, the CXCR-2 inhibitor is compound 1, 2, 3, or 4, or the pharmaceutically acceptable salt thereof. In specific embodiments, the CXCR-2 inhibitor is compound 4 or the pharmaceutically acceptable salt thereof.

Also described herein are methods for treating an acute gout flare by concomitantly or sequentially administering to a subject in need thereof a combination of (i) Colchicine; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof.

In some embodiments, the methods comprise administering less than 1.2 mg colchicine. In some embodiments, the methods comprise administering less than 0.6 mg colchicine. In some embodiments, the methods comprise administering less than 1.2 mg, 1.1 mg, 1.0 mg, 0.9 mg, 0.8 mg, 0.7 mg, 0.6 mg, 0.5 mg, 0.4 mg, 0.3 mg, 0.2 mg, or 0.1 mg colchicine. In some embodiments, the methods comprise administering about 1.2 mg, about 1.1 mg, about 1.0 mg, about 0.9 mg, about 0.8 mg, about 0.7 mg, about 0.6 mg, about 0.5 mg, about 0.4 mg, about 0.3 mg, about 0.2 mg, or about 0.1 mg colchicine. In some embodiments, the methods comprise administering about 0.05 to 0.55 mg colchicine. In some embodiments, the methods comprise administering about 0.2 to 0.4 mg colchicine. In some embodiments, the methods comprise administering about 0.1 to 0.3 mg colchicine.

Also described herein are methods for preventing a gout flare by concomitantly or sequentially administering to a subject in need thereof a combination of (i) Colchicine; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof.

In some embodiments, the methods comprise administering less than 1.2 mg colchicine. In some embodiments, the methods comprise administering less than 0.6 mg colchicine. In some embodiments, the methods comprise administering less than 1.2 mg, 1.1 mg, 1.0 mg, 0.9 mg, 0.8 mg, 0.7 mg, 0.6 mg, 0.5 mg, 0.4 mg, 0.3 mg, 0.2 mg, or 0.1 mg colchicine. In some embodiments, the methods comprise administering about 1.2 mg, about 1.1 mg, about 1.0 mg, about 0.9 mg, about 0.8 mg, about 0.7 mg, about 0.6 mg, about 0.5 mg, about 0.4 mg, about 0.3 mg, about 0.2 mg, or about 0.1 mg colchicine. In some embodiments, the methods comprise administering about 0.05 to 0.55 mg colchicine. In some embodiments, the methods comprise administering about 0.2 to 0.4 mg colchicine. In some embodiments, the methods comprise administering about 0.1 to 0.3 mg colchicine.

Also described herein are methods for improving the therapeutic index of colchicine in a subject by concomitantly or sequentially administering to a subject in need thereof a combination of (i) Colchicine; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof.

In some embodiments, the methods comprise administering less than 1.2 mg colchicine. In some embodiments, the methods comprise administering less than 0.6 mg colchicine. In some embodiments, the methods comprise administering less than 1.2 mg, 1.1 mg, 1.0 mg, 0.9 mg, 0.8 mg, 0.7 mg, 0.6 mg, 0.5 mg, 0.4 mg, 0.3 mg, 0.2 mg, or 0.1 mg colchicine. In some embodiments, the methods comprise administering about 1.2 mg, about 1.1 mg, about 1.0 mg, about 0.9 mg, about 0.8 mg, about 0.7 mg, about 0.6 mg, about 0.5 mg, about 0.4 mg, about 0.3 mg, about 0.2 mg, or about 0.1 mg colchicine. In some embodiments, the methods comprise administering about 0.05 to 0.55 mg colchicine. In some embodiments, the methods comprise administering about 0.2 to 0.4 mg colchicine. In some embodiments, the methods comprise administering about 0.1 to 0.3 mg colchicine.

Also described herein are methods for the prophylaxis and treatment of gout flares in a subject by concomitantly or sequentially administering to a subject in need thereof a combination of (i) Colchicine; and (ii) a CXCR-2 inhibitor. In some embodiments, the subject is an adult. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof.

In some embodiments, the methods comprise administering less than 1.2 mg colchicine. In some embodiments, the methods comprise administering less than 0.6 mg colchicine. In some embodiments, the methods comprise administering less than 1.2 mg, 1.1 mg, 1.0 mg, 0.9 mg, 0.8 mg, 0.7 mg, 0.6 mg, 0.5 mg, 0.4 mg, 0.3 mg, 0.2 mg, or 0.1 mg colchicine. In some embodiments, the methods comprise administering about 1.2 mg, about 1.1 mg, about 1.0 mg, about 0.9 mg, about 0.8 mg, about 0.7 mg, about 0.6 mg, about 0.5 mg, about 0.4 mg, about 0.3 mg, about 0.2 mg, or about 0.1 mg colchicine. In some embodiments, the methods comprise administering about 0.05 to 0.55 mg colchicine. In some embodiments, the methods comprise administering about 0.2 to 0.4 mg colchicine. In some embodiments, the methods comprise administering about 0.1 to 0.3 mg colchicine.

Also described herein are combination therapies wherein any one of the CXCR-2 inhibitors disclosed herein, or pharmaceutically acceptable salts thereof, is administered concurrently or sequentially with an additional therapy and/or an agent for the treatment of an IL-1- and/or MPO-mediated disease or disorder. In some embodiments, a CXCR-2 inhibitor, or pharmaceutically acceptable salt thereof, is administered concurrently or sequentially with an additional an agent for the treatment of an IL-1- and/or MPO-mediated disease or disorder. In some embodiments, the CXCR-2 inhibitor, or pharmaceutically acceptable salts thereof, is administered concurrently or sequentially with colchicine. In some embodiments, the CXCR-2 inhibitor is compound 1, 2, 3, or 4, or the pharmaceutically acceptable salt thereof. In specific embodiments, the CXCR-2 inhibitor is compound 4 or the pharmaceutically acceptable salt thereof.

Also described herein are methods for treating an IL-1- and/or MPO-mediated disease or disorder by concomitantly or sequentially administering to a subject in need thereof a combination of (i) Colchicine; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof.

In some embodiments, the methods comprise administering less than 1.2 mg colchicine. In some embodiments, the methods comprise administering less than 0.6 mg colchicine. In some embodiments, the methods comprise administering less than 1.2 mg, 1.1 mg, 1.0 mg, 0.9 mg, 0.8 mg, 0.7 mg, 0.6 mg, 0.5 mg, 0.4 mg, 0.3 mg, 0.2 mg, or 0.1 mg colchicine. In some embodiments, the methods comprise administering about 1.2 mg, about 1.1 mg, about 1.0 mg, about 0.9 mg, about 0.8 mg, about 0.7 mg, about 0.6 mg, about 0.5 mg, about 0.4 mg, about 0.3 mg, about 0.2 mg, or about 0.1 mg colchicine. In some embodiments, the methods comprise administering about 0.05 to 0.55 mg colchicine. In some embodiments, the methods comprise administering about 0.2 to 0.4 mg colchicine. In some embodiments, the methods comprise administering about 0.1 to 0.3 mg colchicine.

In some embodiments, administration of a combination of colchicine and a CXCR-2 inhibitor provides a synergistic effect. As used herein, the terms “synergy,” “synergistically,” “synergistic” or other grammatical equivalents thereof refer to a combination of therapies (e.g., colchicine and a CXCR-2 inhibitor) that is more effective than the expected additive effects of any two or more single therapies. For example, a synergistic effect of a combination of therapies permits the use of lower dosages of one or more of the therapies and/or less frequent administration of said therapies to a subject. The ability to utilize lower dosages of therapies and/or to administer the therapies less frequently reduces the toxicity associated with the administration of the therapies to a subject without reducing the efficacy of said therapies in the prevention, management, treatment, or amelioration of a given disease, such as an IL-1- or MPO-mediated disease. In addition, a synergistic effect can result in improved efficacy of therapies in the prevention, management, treatment, or amelioration of a given disease, such as an IL-1- or MPO-mediated disease. Synergistic effects of a combination of therapies may avoid or reduce adverse or unwanted side effects associated with the use of any single therapy. The “synergy,” “synergism,” or “synergistic” effect of a combination may be determined herein by the methods of Chou et al., and/or Clarke et al. See Ting-Chao Chou, Theoretical Basis, Experimental Design, and Computerized Simulation of Synergism and Antagonism in Drug Combination Studies, Pharmacol Rev 58:621-681 (2006), and Clarke et al., Issues in experimental design and endpoint analysis in the study of experimental cytotoxic agents in vivo in breast cancer and other models, Breast Cancer Research and Treatment 46:255-278 (1997), which are both incorporated by reference for the methods of determining the “synergy,” synergism,” or “synergistic” effect of a combination.

In some embodiments, the co-administration of the CXCR-2 inhibitor, such as compound 3 or compound 4, or pharmaceutically acceptable salts thereof, results in the need for a smaller dose of a second active agent (e.g., colchicine). In some embodiments, the co-administration of a second active agent (e.g., colchicine) and the CXCR-2 inhibitor, such as compound 3 or compound 4, or pharmaceutically acceptable salts thereof, results in the need for a smaller dose of the CXCR-2 inhibitor, such as compound 3 or compound 4, or pharmaceutically acceptable salts thereof, to treat a disease or disorder. In some embodiments, the co-administration of the CXCR-2 inhibitor, such as compound 3 or compound 4, or pharmaceutically acceptable salts thereof, results in the need for a smaller dose of colchicine to treat or prevent a gout flare. In some embodiments, the co-administration of the CXCR-2 inhibitor, such as compound 3 or compound 4, or pharmaceutically acceptable salts thereof, results in the need for a smaller dose of colchicine to treat or prevent an IL-1 or MPO-mediated disease or disorder. In some embodiments, the smaller dose of colchicine and/CXCR-2 inhibitor is a sub-therapeutically effective amount.

It is difficult to predict the effect of many combination therapies. For example, some drugs interact with each other to reduce therapeutic effectiveness or cause undesired side-effects. These drugs are typically categorized as having an antagonistic effect. Other drug combinations manifest their therapeutic effectiveness as the sum of individual drugs. These combinations are categorized as having an additive effect. Still other drug combinations result in a therapeutic index that is greater than the sum of individual drugs. These are categorized as having a synergistic effect.

Combination therapies having a synergistic effect are highly desirable for many reasons. In some instances, each component in the synergistic combination therapy is used in an amount lower than the therapeutic amount of each individual drug in monotherapy (i.e., single drug administration). Moreover, the risk and/or the severity of side-effects can be reduced significantly by reducing the amount of each drug. Furthermore, combination therapy may significantly increase the overall effectiveness of treatment.

Synergistic actions of combination therapy are particularly useful in treatments where the side-effects are extreme or severe and/or where the efficacy of monotherapy is less than desirable.

### Pharmaceutical Compositions

The compound, compound forms and compositions described herein are administered either alone, or in combination with, pharmaceutically acceptable adjuvants, carriers, excipients, or diluents in a pharmaceutical composition, according to standard pharmaceutical practice.

Described herein in some embodiments are pharmaceutical compositions comprising a CXCR-2 inhibitor, such as any one of the compounds described herein, or the pharmaceutically acceptable salt thereof in association with a pharmaceutically acceptable adjuvant, diluent or carrier. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof. In some embodiments, the pharmaceutical compositions further comprise colchicine.

Also described herein in some embodiments are pharmaceutical compositions comprising colchicine and a CXCR-2 inhibitor in association with a pharmaceutically acceptable adjuvant, diluent or carrier. Also disclosed herein are pharmaceutical compositions comprising a therapeutically-effective amount of Colchicine, and a therapeutically-effective amount of a CXCR-2 inhibitor. Also disclosed herein are pharmaceutical compositions comprising a sub-therapeutically-effective amount of Colchicine, and a sub-therapeutically-effective amount of a CXCR-2 inhibitor. In some embodiments, the pharmaceutical compositions have a fixed dose combination. In some embodiments, the pharmaceutical compositions comprise from about 0.1 mg to about 0.5 mg Colchicine; and a CXCR-2 inhibitor. In some embodiments, the pharmaceutical compositions comprise about 0.1 mg, about 0.2 mg, about 0.3 mg, about 0.4 mg, or about 0.5 mg Colchicine; and a CXCR-2 inhibitor. In some embodiments, the pharmaceutical compositions comprise from about 0.1 mg to about 0.6 mg Colchicine; and a CXCR-2 inhibitor. In some embodiments, the pharmaceutical compositions comprise about 0.1 mg, about 0.2 mg, about 0.3 mg, about 0.4 mg, about 0.5 mg, or about 0.6 mg Colchicine; and a CXCR-2 inhibitor. In some embodiments, the pharmaceutical compositions comprise from about 0.1 mg to about 1.0 mg Colchicine; and a CXCR-2 inhibitor. In some embodiments, the pharmaceutical compositions comprise about 0.1 mg, about 0.2 mg, about 0.3 mg, about 0.4 mg, about 0.5 mg, about 0.6 mg, about 0.7 mg, about 0.8 mg, about 0.9 mg, or 1.0 mg Colchicine; and a CXCR-2 inhibitor. In some embodiments, the pharmaceutical compositions comprise less than 0.5 mg of Colchicine; and a CXCR-2 inhibitor. In some embodiments, the pharmaceutical compositions further comprise a pharmaceutically acceptable diluent or carrier.

In some embodiments, the CXCR-2 inhibitor is one of the compounds disclosed herein, or a pharmaceutically acceptable salt thereof. In some embodiments, the CXCR-2 inhibitor is 1-(4-chloro-2-hydroxy-3-(piperazin-1-ylsulfonyl)phenyl)-3-(2-chloro-3-fluorophenyl)urea (compound 1), or a pharmaceutically acceptable salt thereof. In some embodiments, the CXCR-2 inhibitor is (R)-2-hydroxy-N,N-dimethyl-3-((2-((1-(5-methylfuran-2-yl)propyl)amino)-3,4-dioxocyclobut-1-en-1-yl)amino)benzamide (compound 2), or a pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4) or a pharmaceutically acceptable salt thereof.

In some embodiments, the pharmaceutical compositions are for the treatment of disorders. In some embodiments, the pharmaceutical compositions are for the treatment of disorders in a mammal. In some embodiments, the pharmaceutical compositions are for the treatment of disorders in a human. In some embodiments, the pharmaceutical compositions are for the treatment or prophylaxis of crystal arthropathy diseases. In some embodiments, the pharmaceutical compositions are for the treatment or prophylaxis of diseases characterized by the accumulation of crystals in one or more joints. In some embodiments, the pharmaceutical compositions are for the treatment or prophylaxis of gout, gouty arthritis, and gout flares.

Depending on the mode of administration, the pharmaceutical composition will conveniently comprise from 0.05 to 99% w (percent by weight), more conveniently from 0.05 to 80% w, still more conveniently from 0.10 to 70% w, and even more conveniently from 0.10 to 50% w, of active ingredient, all percentages by weight being based on total composition.

Also described herein are processes for the preparation of a pharmaceutical composition of the invention which comprise mixing a CXCR-2 inhibitor (e.g., compound 3 or 4), or pharmaceutically acceptable salts thereof, with a pharmaceutically acceptable adjuvant, diluent or carrier. In some embodiments, the pharmaceutical compositions are administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Conveniently, compound 3 or compound 4, or pharmaceutically acceptable salts thereof, is administered orally.

### Modes of Administration

The compound, compound forms and compositions described herein are administered either alone, or in combination with, pharmaceutically acceptable adjuvants, carriers, excipients, or diluents in a pharmaceutical composition, according to standard pharmaceutical practice.

The pharmaceutical compositions described herein are, for example, in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition is, in some embodiments, in unit dosage forms suitable for single administration of precise dosages. Pharmaceutical compositions include a compound or compound form as described herein as an active ingredient, and a conventional pharmaceutical carrier or excipient. In some embodiments, these compositions include other or additional medicinal or pharmaceutical agents, carriers, adjuvants, etc.

Pharmaceutical compositions are conveniently presented in unit dosage form. In some embodiments, they are prepared with a specific amount of active compound by any of the methods well known or apparent to those skilled in the pharmaceutical arts.

### Doses

The amount of pharmaceutical compositions administered will firstly be dependent on the mammal being treated. In the instances where pharmaceutical compositions are administered to a human subject, the daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, drug combination, and the discretion of the prescribing physician. Also, the route of administration varies depending on the condition and its severity. The pharmaceutical composition is, in some embodiments, in unit dosage form. In such form, the preparation is subdivided into unit doses containing appropriate quantities of the active component, e.g., an effective amount to achieve the desired purpose. Determination of the proper dosage for a particular situation is within the skill of the art. For convenience, in some embodiments, the total daily dosage is divided and administered in portions during the day if desired. The amount and frequency of administration will be regulated according to the judgment of the attending clinician physician considering such factors as described above. Thus, the amount of pharmaceutical composition to be administered is variable depending upon the circumstances. In some instances, dosage levels below the lower limit of the aforesaid range are more than adequate, while in other cases still larger doses are employed without causing any harmful side effect, e.g. by dividing such larger doses into several small doses for administration throughout the day. In combinational applications in which the compound is not the sole therapy, it is possible to administer lesser amounts of compound and still have therapeutic or prophylactic effect.

### Kits

The compounds, compound forms, compositions and methods described herein provide kits for the treatment of diseases and disorders, such as the ones described herein. These kits comprise a compound, compound form, compounds, compound forms or compositions described herein in a container and, optionally, instructions teaching the use of the kit according to the various methods and approaches described herein. Such kits, in some embodiments, also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein are provided, marketed and/or promoted to health providers, including physicians, nurses, pharmacists, formulary officials, and the like. Kits are also, in some embodiments, marketed directly to the consumer.

Described herein are compositions or kits comprising a CXCR-2 inhibitor, such as any one of the compounds described herein, or the pharmaceutically acceptable salt thereof. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof. In some embodiments, the kits further comprise Colchicine.

Described herein are compositions or kits for treating a subject experiencing a gout flare comprising a CXCR-2 inhibitor, such as any one of the compounds described herein, and instructions for administration of the CXCR-2 inhibitor to treat the gout flare. In some embodiments, the CXCR-2 inhibitor is N-(2-((2,3-difluorobenzyl)thio)-6-(((2R,3S)-3,4-dihydroxybutan-2-yl)oxy)pyrimidin-4-yl)azetidine-1-sulfonamide (compound 3) or a pharmaceutically acceptable salt thereof. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide (compound 4), or a pharmaceutically acceptable salt thereof. In some embodiments, the kits further comprise Colchicine.

Provided in certain embodiments, are compositions or kits comprising a CXCR-2 inhibitor, a double low density polyethylene plastic bag, and an HDPE container. In further embodiments, the composition or kit further comprises a foil bag (e.g., an anhydrous foil bag, such as a heat sealed anhydrous foil bag). In some embodiments, the composition or kit further comprises a desiccant; in still other embodiments, a desiccant is not necessary and/or present. In some instances, such packing improves the stability of the CXCR-2 inhibitor.

In some embodiments, the compounds, compound forms and pharmaceutical compositions described herein are utilized for diagnostics and as research reagents. For example, in some embodiments, the compounds, compound forms and pharmaceutical compositions, either alone or in combination with other compounds, are used as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. These analyses are performed on stimulated or unstimulated cells and in the presence or absence of other compounds which affect expression patterns.

Besides being useful for human treatment, the compounds, compound forms and pharmaceutical compositions described herein are also useful for veterinary treatment of animals.

The examples and preparations provided below further illustrate and exemplify the compounds of the present invention and methods of preparing such compounds. It is to be understood that the scope of the present invention is not limited in any way by the scope of the following examples and preparations.

### EXAMPLES

The following examples further illustrate the invention but should not be construed as in any way limiting its scope. In particular, the processing conditions are merely exemplary and can be readily varied by one of ordinary skill in the art.

All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Unless defined otherwise, technical and scientific terms used herein have the same meaning as is commonly understood by one of skill in the art to which this invention belongs.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited herein. Moreover, any combination of the above-described elements in all possible variations thereof is encompassed by the invention unless otherwise indicated herein or otherwise clearly contradicted by context.

### Example 1: Test Compounds

Compounds 1, 2, 3 and 4, alone or in combination with colchicine, were tested in disease models of crystal-induced arthropathy.

| No. | Structure | Chemical Name | MW |
|---|---|---|---|
| 1 | 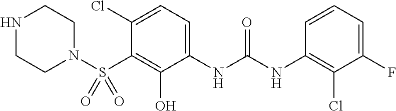 [SMILES: O=C(Nc1ccc(Cl)c(S(=O)(=O)N2CCNCC2)c1O)Nc1cccc(F)c1Cl] | 1-(4-chloro-2-hydroxy-3- (piperazin-1- ylsulfonyl)phenyl)-3-(2- chloro-3-fluorophenyl)urea | 463.3 |
| 2 | 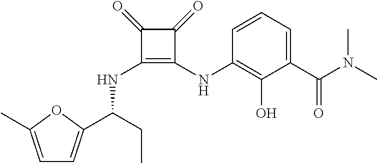 | (R)-2-hydroxy-N,N-dimethyl- 3-((2-((1-(5-methylfuran-2- yl)propyl)amino)-3,4- dioxocyclobut-1-en-1- yl)amino)benzamide | 397.4 |
| 3 | 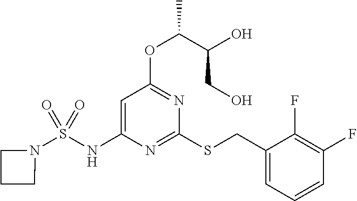 [SMILES: C[C@@H](Oc1cc(NS(=O)(=O)N2CCC2)nc(SCc2cccc(F)c2F)n1)[C@@H](O)CO] | N-(2-((2,3- difluorobenzyl)thio)-6- (((2R,3S)-3,4- dihydroxybutan-2- yl)oxy)pyrimidin-4- yl)azetidine-1-sulfonamide | 476.5 |
| 4 | 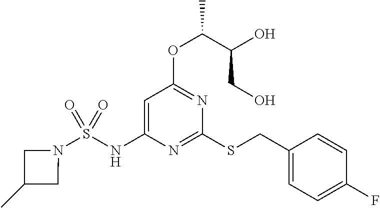 [SMILES: CC1CN(S(=O)(=O)Nc2cc(O[C@H](C)[C@@H](O)CO)nc(SCc3ccc(F)cc3)n2)C1] | N-(6-(((2R,3S)-3,4-dihydroxy butan-2-yl)oxy)-2-((4-fluoro benzyl)thio)pyrimidin-4-yl)-3- methylazetidine-1- sulfonamide | 472.6 |
| | 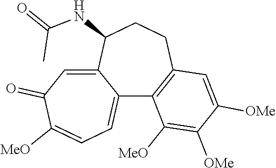 [SMILES: COc1cc2c(c(OC)c1OC)-c1ccc(OC)c(=O)cc1[C@@H](NC(C)=O)CC2] | COLCHICINE | 399.4 |

Colchicine was obtained from Sigma Aldrich. Compounds 1, 2, 3, and 4 were shown to inhibit CXCR-2 (see Dwyer & Yu, Expert Opin. Ther. Patents (2014), 24(5), 519). Compound 1 has been described previously (see for example WO 2009/039091) and was obtained from R&D Systems. Compound 2 was previously described (see for example WO 2009/073683) and was obtained from Medchem Express. Compound 3 was previously described and was prepared as shown in U.S. Pat. No. 8,748,603. Compound 4 was prepared as shown in Scheme 1 (below) and in U.S. Pat. No. 8,735,413.

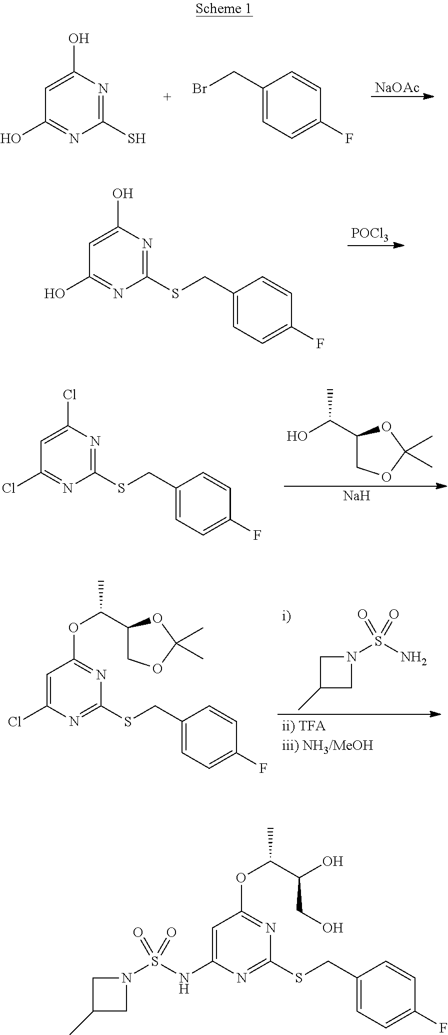

### Example 2: Cell Migration Assay (In Vitro Inflammation)

### Example 2A: Monocyte Isolation from Leukopacks

Primary blood monocytes (PMBC) were obtained from the lymphocyte layer (buffy coat) of peripheral blood from normal donors. Each leukopack (Interstate Blood Bank) was diluted with an equal volume of PBS, and 35 mL of blood preparation was over-laid onto 15 mL of Ficoll-Paque™ PLUS (GE Healthcare Bio-Sciences). Tubes were centrifuged at 700×g without breaks for 30 minutes at room temperature.

The buffy coat interface was removed, added to PBS (40 mL), and centrifuged at 300×g. Any remaining erythrocytes in the cell pellet were lysed by incubating cells in red blood cell lysis buffer (10 mL, R&D Systems) for 10 minutes at room temperature. Following lysis, PBS (40 mL) was added and the cells centrifuged for 5 minutes at 200×g. The pellet was washed once with PBS and re-suspended in complete RPMI and seeded onto a BD Falcon™ 100 mm tissue culture dish (Cat. No. 353003). After one hour, the media was aspirated and the adherent cells (predominantly monocytes) were harvested using a cell scraper and used in chemotaxis assays.

### Example 2B: Chemotaxis Assay

Chemotaxis was assayed in 48-well plates with BD Falcon FluoroBlok Multiwell inserts with 3 μm pores (Cat. No. 351161 or 351162) coated with hFN. Briefly, freshly isolated monocytes were re-suspended in chemotaxis assay buffer (HBSS supplemented with 0.1% BSA) at a density of 2×106 cells/ml. Cells were labeled with 1.0 μM Calcein AM for 40 minutes at 37° C., 5% CO₂. Following incubation, cells were washed once and re-suspended in assay buffer at a density of 2.0×10⁶ cells/ml. Labeled cell suspension was added onto inserts (250 μl/well) and set aside. In a separate BD Falcon™ 48 well, flat-bottom plate, compound 1 or compound 2, (750 μl, 10 Mm) was added. The multiwell insert containing cells was gently lowered into the plate containing chemoattractant and immediately placed into a bottom fluorescence plate reader. Fluorescence emitted from cells that had migrated to the bottom surface of the insert was measured at various time points. Cells labeled with Calcein AM were read at 485/530 nm (Ex/Em) wavelength.

Monosodium urate crystal conditioned media was used. Compound 1 used at 10 μM. Compound 2 used at 10 μM.

### Example 2C: Results

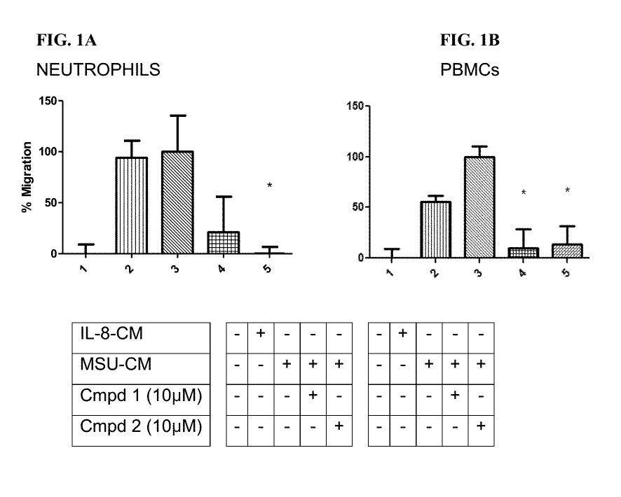

The neutrophil counts are presented graphically in FIG. 1A, which shows Compound 1 and Compound 2 providing significant inhibition of migration in neutrophils (in the MSU-conditioned media). The PBMC counts are presented graphically in FIG. 1B, which shows Compounds 1 and 2 providing significant inhibition in migration in PBMCs.

### Example 3: General Procedures for Rat Air Pouch Model of Crystal-Induced Arthropathy

### Example 3A: Preparation of Reagents

Mono Sodium Urate (MSU):

Sodium hydroxide (40 g, Fisher Scientific) was dissolved in de-ionized water (100 mL, dH₂O) to provide a 10N solution. Uric acid (16 g, Sigma) was added to de-ionized water (3400 mL) containing sodium hydroxide solution (11.8 mL, 10N) and heated to 60° C. with constant stirring. The pH was adjusted to 8.9 with 10N sodium hydroxide solution. The resulting clear solution was cooled to 4-8° C., resulting in the formation of crystals, which were isolated by filtration, washed three times with de-ionized water (1 L) and dried at 37° C. The dried mono sodium urate crystals were sifted into an air-tight container for storage. Mono sodium urate (10 g) was suspended in sterile saline (1 L, 0.9%, USP, Hospira) for injection and placed on a stir plate to maintain a constant 10 mg/mL homogenous suspension.

Vehicle:

Methylcellulose (0.4 g, Sigma) was dissolved in de-ionized water (100 mL) to provide a 0.4% solution used as vehicle.

Colchicine:

Colchicine (7 mg, Sigma) was dissolved in sterile saline (7 mL) to provide a 1 mg/mL solution.

Heparinized Saline:

Heparinized saline (10 U/mL) was prepared by adding heparin (0.4 mL, 10,000 U/ml, APP Pharmaceuticals) to sterile sodium chloride solution (400 mL, 0.9%).

Test Compounds:

Test compounds were suspended in vehicle to provide the desired concentrations, and diluted accordingly. For example, test compound (102.5 mg) was suspended in vehicle (3.417 mL) to provide a 30 mg/mL suspension. 0.3 mL of the 30 mg/mL suspension was added to vehicle (2.7 mL) to provide a 3 mg/mL suspension. 0.3 mL of the 3 mg/mL suspension was added to vehicle (2.7 mL) to provide a 0.3 mg/mL suspension.

### Example 3B: Rats

Male Sprague-Dawley rats (Charles River Laboratories, 160-180 g) were received, individually examined and housed in cages of five rats each. The animals were in apparent good health with no clinical signs of disease or distress. The rats were placed in quarantine with daily inspections, ear notched for identification purposes and shaved at the nape of the neck.

Day 0:

The rats were anesthetized (isoflurane), and the nape of the neck was cleansed with 70% isopropanol (Butler Animal Health Supply) followed by povidone-iodine solution (Ricca Chemical Co.). Sterile air (30 mL, 0.2 μm, Millipore) was injected subcutaneously using a 23G×1½ inch needle fixed to a 30 mL syringe. The rats were returned to routine housing with no adverse reactions observed.

Day 3:

The rats were anesthetized (isoflurane), and the nape of the neck was cleansed with 70% isopropanol (Butler Animal Health Supply) followed by povidone-iodine solution (Ricca Chemical Co.). Sterile air (15 mL, 0.11 μm, Millipore) was injected subcutaneously using a 23G×1½ inch needle fixed to a 20 mL syringe. The rats were returned to routine housing with no adverse reactions observed.

The rats were weighed and sorted into treatment groups based on average body weight.

As appropriate, the rats were dosed orally with test compound or vehicle (saline alone).

As appropriate, the rats were injected subcutaneously with colchicine (1 mL/kg).

As appropriate, the rats were dosed with test compound (oral administration) in combination with colchicine (injected subcutaneously).

Thirty minutes after SC injection or two hours after PO dosing, MSU (15 mL) was injected into the air pouch using an 18G×2 inch needle fitted to a 20 mL syringe. Control group was injected with 15 mL sterile saline (vehicle). The injection sites were closed (collodion, Macron) and the rats returned to their cages with no adverse effects observed.

### Example 3C: Samples

Four hours after MSU/saline injection, the rats were anesthetized and heparinized saline (5 mL, 10 U/mL) was injected into the air pouch. The air pouch was gently massaged, the contents immediately removed using a 14G×1 inch needle fitted to a 6 mL syringe, and the exudate volume recorded. An aliquot of the exudate was transferred to green Eppendorf tubes for total white blood cell (WBC) measurement. After allowing the MSU crystals to settle out for ten minutes, an aliquot of the exudate was transferred to heparinized microtainer tubes (Becton Dickinson) for differential white blood cell counts. The remainder of the exudate was centrifuged and an aliquot of the supernatants was dispensed to labeled clear Eppendorf tubes and stored at −80° C. The rats were exsanguinated into pre-chilled serum separator tubes, processed to serum, and 0.5 mL aliquot was stored at −80° C. in labeled Eppendorf tubes.

### Example 4: Compound 2 in Rat Air Pouch Model

Compound 2 was tested according to the protocol described in example 2. 60 rats were used, divided into 6 groups of 10 animals, as follows:

| | Group | | | | | |
|---|---|---|---|---|---|---|
| | 1 | 2 | 3 | 4 | 5 | 6 |
| Treat- | Vehicle | | Colchi- | Compound 2 | | |
| ment | | | cine | | | |
| *ROA | PO | PO | SC | PO | PO | PO |
| | | | (−2 Hr) | | | |
| Dose | 1 ml/kg | 1 ml/kg | 1 mg/kg | 0.3 mg/kg | 3.0 mg/kg | 30 mg/kg |

*ROA = route of administration − oral (PO) or subcutaneous injection (SC)

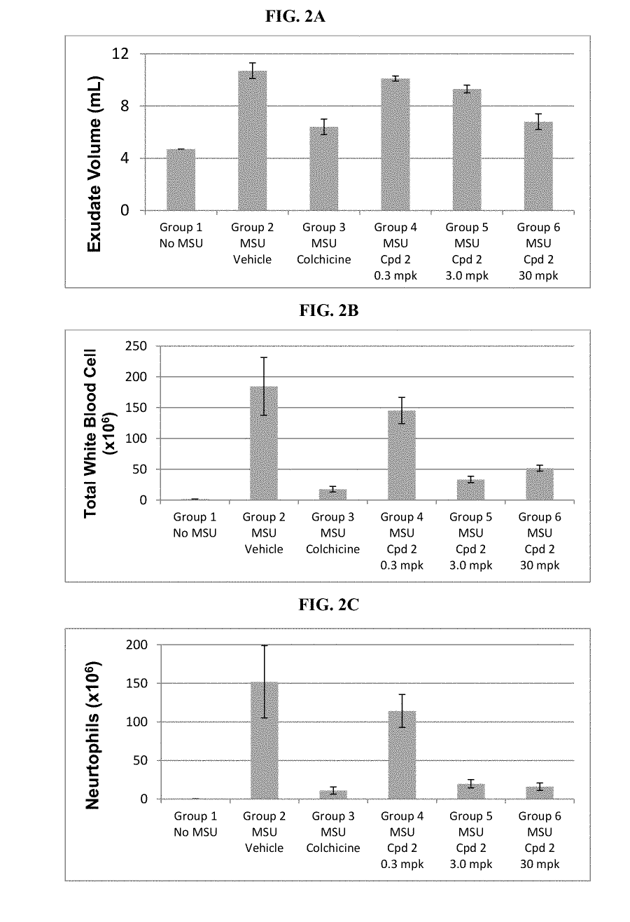

The results—average exudate volume (FIG. 2A), total white blood cell counts (FIG. 2B) and neutrophil counts (FIG. 2C) are provided in the table below and presented in FIG. 2.

| | Group 1 | Group 2 | Group 3 | Group 4 | Group 5 | Group 6 |
|---|---|---|---|---|---|---|
| MSU | N | Y | Y | Y | Y | Y |
| Treatment | Vehicle | | Colchicine | | Compound 2 | |
| Route of Admin. | | | SC | PO | PO | PO |
| Dose | | | 1 mg/kg | 0.3 mg/kg | 3 mg/kg | 30 mg/kg |
| Avg Exudate Vol (mL) | 4.7 | 10.7 | ***6.4 | 10.1 | 9.3 | ***6.8 |
| SE | 0.0 | 0.6 | 0.6 | 0.2 | 0.3 | 0.6 |
| Avg Total WBC (×10⁶) | 1.7 | 184.5 | **17.6 | 145.2 | *33.4 | *51.7 |
| SE (×10⁶) | 0.6 | 54.3 | 7.2 | 27.2 | 6.9 | 11.9 |
| Neutrophils (×10⁶) | 0.3 | 152.0 | **11.0 | 114.2 | *19.8 | **16.0 |
| SE (×10⁶) | 0.10 | 46.86 | 4.64 | 21.34 | 5.32 | 4.81 |

p-test:
***= p < 0.001;
**= p < 0.05;
*= p < 0.01

### Example 5: Compound 2 in Combination with Colchicine in the Rat Air Pouch Model

Compound 2 was tested, in the presence and absence of colchicine, at various doses, according to the protocol described in Example 2. 90 rats were used, divided into 9 groups of 10 animals, as follows:

| Group | Treatment | Dose | | ROA |
|---|---|---|---|---|
| 1 | Vehicle | 1 | ml/kg | PO |
| 2 | Vehicle | 1 | ml/kg | PO |
| 3 | Colchicine | 0.1 | mg/kg | PO |
| 4 | Cpd 2 | 0.3 | mg/kg | PO |
| 5 | Cpd 2 | 3.0 | mg/kg | PO |
| 6 | Cpd 2 | 0.3 | mg/kg | PO |
| | Colchicine | 0.01 | mg/kg | SC |
| 7 | Cpd 2 | 3.0 | mg/kg | PO |
| | Colchicine | 0.01 | mg/kg | SC |
| 8 | Cpd 2 | 0.3 | mg/kg | PO |
| | Colchicine | 0.1 | mg/kg | SC |
| 9 | Cpd 2 | 3.0 | mg/kg | PO |
| | Colchicine | 0.1 | mg/kg | SC |

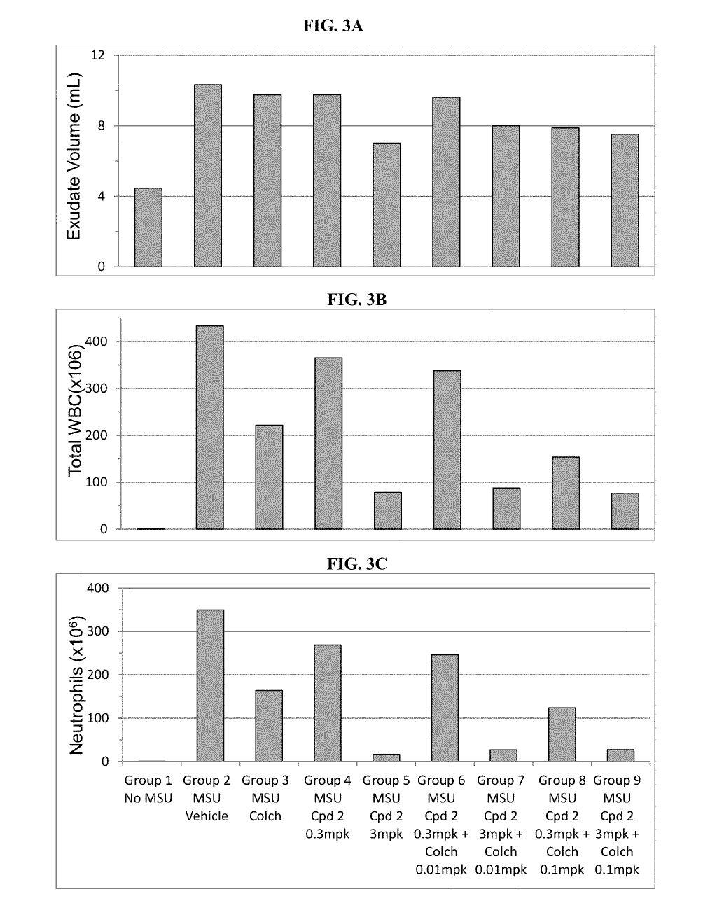

The results—average exudate volume (FIG. 3A), total white blood cell counts (FIG. 3B) and neutrophil counts (FIG. 3C) are provided in the table below and presented in FIGS. 3A-3C.

| | | | | | Avg Exudate | | Avg Total | | Neutrophils | |
|---|---|---|---|---|---|---|---|---|---|---|
| Gp | MSU | Treatment | Dose | | Vol (mL) | SE | WBC (×10⁶) | SE | (×10⁶) | SE |
| 1 | N | Vehicle | | | 4.47 | 0.2 | 0.7 | 0.6 | 0.2 | 0.3 |
| 2 | Y | Vehicle | | | 10.3 | 1.4 | 433 | 492.8 | 349 | 442 |
| 3 | Y | Colchicine | 0.1 | mpk | 9.7 | 0.6 | 221 | 52.1 | 163 | 55.9 |
| 4 | Y | Cpd 2 | 0.3 | umol/kg | 9.8 | 1.1 | 365 | 249.2 | 268 | 182.7 |
| 5 | Y | Cpd 2 | 3.0 | umol/kg | 7.0 | 2.3 | 78.6 | 124.4 | 16 | 23.2 |
| 6 | Y | Cpd 2 | 0.3 | umol/kg | 9.6 | 1.8 | 337 | 282.4 | 246 | 216.9 |
| | | Colchicine | 0.001 | mpk | | | | | | |
| 7 | Y | Cpd 2 | 3.0 | umol/kg | 8 | 1.2 | 88 | 72.4 | 26.7 | 15.8 |
| | | Colchicine | 0.001 | mpk | | | | | | |
| 8 | Y | Cpd 2 | 0.3 | umol/kg | 7.9 | 1.7 | 153 | 97.2 | 123 | 77.5 |
| | | Colchicine | 0.1 | mpk | | | | | | |
| 9 | Y | Cpd 2 | 3.0 | umol/kg | 7.5 | 1.3 | 76.8 | 47.6 | 27.2 | 32 |
| | | Colchicine | 0.1 | mpk | | | | | | |

### Example 6: Compound 2, Compound 3 & Compound 4 in the Rat Air Pouch Model

Compounds 2, 3 and 4 were tested according to the protocol described in Example 2. 100 rats were used, divided into 10 groups of 10 animals, as follows:

| Group | Treatment | Dose | | ROA |
|---|---|---|---|---|
| 1 | Vehicle | 10 | ml/kg | PO |
| 2 | Vehicle | 10 | ml/kg | PO |
| 3 | Cpd 2 | 3 | mg/kg | PO |
| 4 | Colchicine | 1 | mg/kg | SC |
| 5 | Cpd 3 | 0.3 | μmole/kg | PO |
| 6 | Cpd 3 | 3 | μmole/kg | PO |
| 7 | Cpd 3 | 30 | μmole/kg | PO |
| 8 | Cpd 4 | 1 | μmole/kg | PO |
| 9 | Cpd 4 | 30 | μmole/kg | PO |
| 10 | Cpd 4 | 100 | μmole/kg | PO |

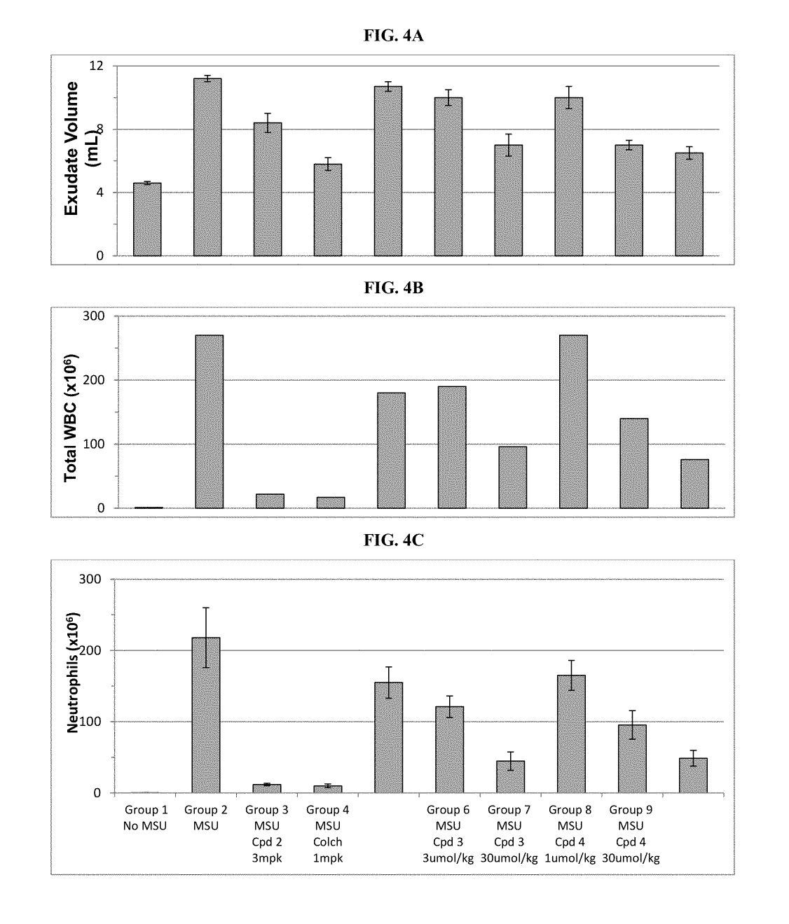

The results—average exudate volume (FIG. 4A), total white blood cell counts (FIG. 4B) and neutrophil counts (FIG. 4C) are provided in the table below and presented in FIGS. 4A-4C.

| | | | | | Avg Exudate | | Avg Total | | Neutrophils | |
|---|---|---|---|---|---|---|---|---|---|---|
| Gp | MSU | Treatment | Dose | | Vol (mL) | SE | WBC (×10⁶) | SE | (×10⁶) | SE |
| 1 | N | Vehicle | | | 4.6 | 0.1 | 1.1 | 0.21 | 0.25 | 0.05 |
| 2 | Y | Vehicle | | | 11.2 | 0.2 | 270 | 45 | 218 | 42 |
| 3 | Y | Cpd 2 | 3 | mpk | 8.4 | 0.6 | 22 | 2.7 | 11.8 | 1.7 |
| 4 | Y | Colchicine | 1 | mpk | 5.8 | 0.4 | 17 | 3.8 | 9.9 | 2.5 |
| 5 | Y | Cpd 3 | 0.3 | umol/kg | 10.7 | 0.3 | 180 | 23 | 155 | 22 |
| 6 | Y | Cpd 3 | 3.0 | umol/kg | 10 | 0.5 | 190 | 21 | 121 | 15 |
| 7 | Y | Cpd 3 | 30.0 | umol/kg | 7 | 0.7 | 96 | 33 | 44.6 | 13 |
| 8 | Y | Cpd 4 | 1.0 | umol/kg | 10 | 0.7 | 270 | 62 | 165 | 21 |
| 9 | Y | Cpd 4 | 30 | umol/kg | 7 | 0.3 | 140 | 27 | 95.4 | 20 |
| 10 | Y | Cpd 4 | 100 | umol/kg | 6.5 | 0.4 | 76 | 19 | 48.6 | 11 |

### Example 7: Compound 3 and Compound 4 in Combination with Colchicine in the Rat Air Pouch Model

Compounds 3 and 4 were tested, in the presence and absence of colchicine, at various doses, according to the protocol described in Example 2. 100 rats were used, divided into 10 groups of 10 animals, as follows:

| Group | Treatment | Dose | | ROA |
|---|---|---|---|---|
| 1 | Vehicle | 10 | ml/kg | PO |
| 2 | Vehicle | 10 | ml/kg | PO |
| 3 | Colchicine | 0.1 | mg/kg | SC |
| 4 | Colchicine | 1.0 | mg/kg | SC |
| 5 | Cpd 3 | 3 | μmole/kg | PO |
| 6 | Cpd 3 | 30 | μmole/kg | PO |
| 7 | Cpd 4 | 1 | μmole/kg | PO |
| 8 | Cpd 4 | 30 | μmole/kg | PO |
| 9 | Cpd 4 | 1 | μmole/kg | PO |
| | Colchicine | 0.1 | mg/kg | SC |
| 10 | Cpd 3 | 3 | μmole/kg | PO |
| | Colchicine | 0.1 | mg/kg | SC |

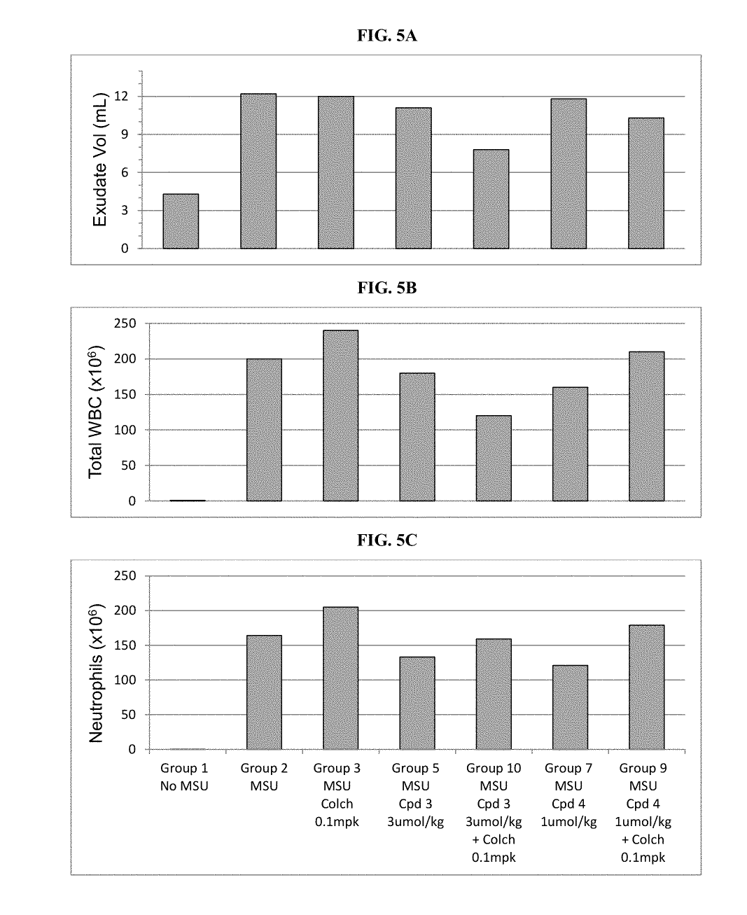

The results—average exudate volume (FIG. 5A), total white blood cell counts (FIG. 5B) and neutrophil counts (FIG. 5C) are provided in the table below and presented in FIGS. 5A-5C.

| | | | | | Avg Exudate | | Avg Total | | Neutrophils | |
|---|---|---|---|---|---|---|---|---|---|---|
| Gp | MSU | Treatment | Dose | | Vol (mL) | SE | WBC (×10⁶) | SE | (×10⁶) | SE |
| 1 | N | Vehicle | | | 4.3 | 0 | 0.9 | 0.2 | 0.2 | 0.06 |
| 2 | Y | Vehicle | | | 12.2 | 0.3 | 200 | 23 | 164 | 20 |
| 3 | Y | Colchicine | 0.1 | mpk | 12 | 0.4 | 240 | 34 | 205 | 32 |
| 4 | Y | Colchicine | 1.0 | mpk | 6 | 0.4 | 22 | 7.7 | 10.8 | 2.9 |
| 5 | Y | Cpd 3 | 3 | umol/kg | 11.1 | 0.4 | 180 | 55 | 133 | 33 |
| 6 | Y | Cpd 3 | 30 | umol/kg | 7.4 | 0.5 | 110 | 28 | 65.2 | 19 |
| 7 | Y | Cpd 4 | 1 | umol/kg | 11.8 | 0.4 | 160 | 30 | 121 | 22 |
| 8 | Y | Cpd 4 | 30 | umol/kg | 8.1 | 0.5 | 100 | 29 | 69.6 | 19 |
| 9 | Y | Cpd 4 | 1 | umol/kg | 10.3 | 0.3 | 210 | 45 | 179 | 41 |
| | | Colchicine | 0.1 | mpk | | | | | | |
| 10 | Y | Cpd 3 | 3.0 | umol/kg | 7.8 | 0.5 | 120 | 34 | 159 | 38 |
| | | Colchicine | 0.1 | mpk | | | | | | |

### Example 8: Compound 3 in Combination with Colchicine in the Rat Air Pouch Model

Compounds 3 was tested, in the presence and absence of colchicine, at various doses, according to the protocol described in Example 2. 100 rats were used, divided into 10 groups of 10 animals, as follows:

| Group | Treatment | Dose | | ROA |
|---|---|---|---|---|
| 1 | Vehicle | 10 | ml/kg | PO |
| 2 | Vehicle | 10 | ml/kg | PO |
| 3 | Colchicine | 0.3 | mg/kg | SC |
| 4 | Colchicine | 0.5 | mg/kg | SC |
| 5 | Colchicine | 1.0 | mg/kg | SC |
| 6 | Cpd 3 | 10 | μmol/kg | PO |
| 7 | Cpd 3 | 30 | μmol/kg | PO |
| 8 | Cpd 3 | 100 | μmol/kg | PO |
| 9 | Cpd 3 | 300 | μmole/kg | PO |
| 10 | Cpd 3 | 10 | μmol/kg | PO |
| | Colchicine | 0.3 | mg/kg | SC |

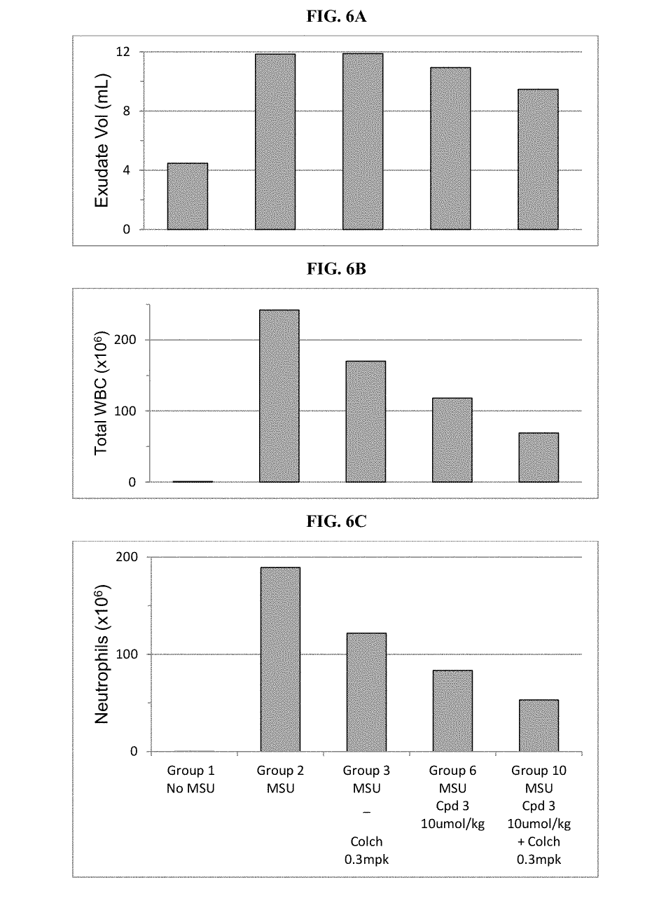

The results—average exudate volume (FIG. 6A), total white blood cell counts (FIG. 6B) and neutrophil counts (FIG. 6C) are provided in the table below and presented in FIGS. 6A-6C (Groups 1, 2, 3, 6 and 10).

| | | | | | Avg Exudate | | Avg Total | | Neutrophils | |
|---|---|---|---|---|---|---|---|---|---|---|
| Gp | MSU | Treatment | Dose | | Vol (mL) | SE | WBC (×10⁶) | SE | (×10⁶) | SE |
| 1 | N | Vehicle | | | 4.47 | 0.1 | 0.8 | 0.2 | 0.14 | 0.05 |
| 2 | Y | Vehicle | | | 11.84 | 0.3 | 242 | 31.9 | 189.3 | 21.4 |
| 3 | Y | Colchicine | 0.3 | mpk | 11.88 | 0.3 | 170 | 64.3 | 121.8 | 51.9 |
| 4 | Y | Colchicine | 0.5 | mpk | 7.62 | 0.1 | 96.3 | 28.2 | 64.6 | 18.2 |
| 5 | Y | Colchicine | 1.0 | mpk | 6.8 | 0.2 | 41.5 | 13.9 | 7.6 | 2.9 |
| 6 | Y | Cpd 3 | 10.0 | umol/kg | 10.93 | 0.4 | 118 | 30.3 | 83.3 | 24.4 |
| 7 | Y | Cpd 3 | 30.0 | umol/kg | 8.42 | 0.3 | 70.5 | 20.3 | 40.3 | 13.6 |
| 8 | Y | Cpd 3 | 100 | umol/kg | 6.56 | 0.2 | 56.1 | 18.8 | 25.2 | 10.5 |
| 9 | Y | Cpd 3 | 300 | umol/kg | 5.7 | 0.3 | 24.7 | 10.7 | 5.3 | 3.8 |
| 10 | Y | Cpd 3 | 10.0 | umol/kg | 9.46 | 0.6 | 69.1 | 26.9 | 53.1 | 20.4 |
| | | Colchicine | 0.3 | mpk | | | | | | |

### Example 9: Compound 4 in Combination with Colchicine in the Rat Air Pouch Model

Compound 4 was tested, in the presence and absence of colchicine, at various doses, according to the protocol described in Example 2. 100 rats were used, divided into 10 groups of 10 animals, as follows:

| Group | Treatment | Dose (mg/kg) | | ROA |
|---|---|---|---|---|
| 1 | Vehicle | 10 | ml/kg | PO |
| 2 | Vehicle | 10 | ml/kg | PO |
| 3 | Colchicine | 0.3 | mg/kg | SC |
| 4 | Colchicine | 0.5 | mg/kg | SC |
| 5 | Colchicine | 1.0 | mg/kg | SC |
| 6 | Cpd 4 | 10 | μmol/kg | PO |
| 7 | Cpd 4 | 30 | μmol/kg | PO |
| 8 | Cpd 4 | 100 | μmol/kg | PO |
| 9 | Cpd 4 | 300 | μmol/kg | PO |
| 10 | Cpd 4 | 10 | μmole/kg | PO |
| | Colchicine | 0.3 | mg/kg | SC |

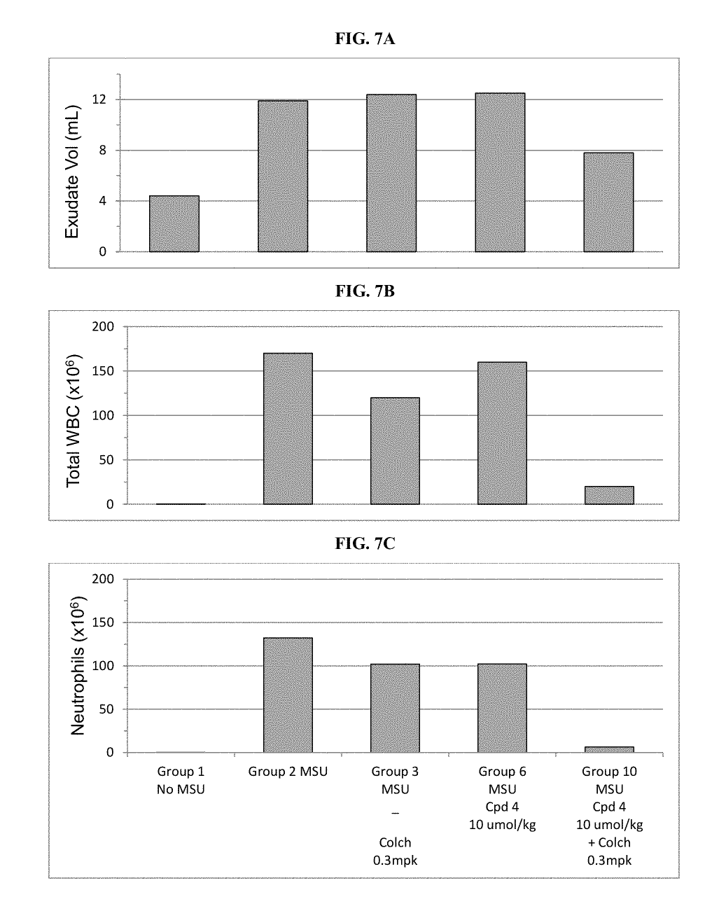

The results—average exudate volume (FIG. 7A), total white blood cell counts (FIG. 7B) and neutrophil counts (FIG. 7C) are provided in the table below and presented in FIGS. 7A-7C (Groups 1, 2, 3, 6 and 10).

| | | | | | Avg Exudate | | Avg Tot | | Neutrophils | |
|---|---|---|---|---|---|---|---|---|---|---|
| Gp | MSU | Treatment | Dose | | Vol (mL) | SE | WBC (×10⁶) | SE | (×10⁶) | SE |
| 1 | N | Vehicle | | | 4.4 | 0.1 | 0.3 | 0.2 | 0.09 | 0.02 |
| 2 | Y | Vehicle | | | 11.9 | 0.5 | 170 | 39 | 132.2 | 34.7 |
| 3 | Y | Colchicine | 0.3 | mpk | 12.4 | 0.4 | 120 | 13.9 | 101.9 | 13.7 |
| 4 | Y | Colchicine | 0.5 | mpk | 8.8 | 0.5 | 58 | 10.6 | 42.7 | 8.8 |
| 5 | Y | Colchicine | 1.0 | mpk | 5.6 | 0.2 | 16 | 5.9 | 9.8 | 4.1 |
| 6 | Y | Cpd 4 | 10.0 | umol/kg | 12.5 | 0.5 | 160 | 40.4 | 102.1 | 29.4 |
| 7 | Y | Cpd 4 | 30.0 | umol/kg | 6.7 | 0.4 | 28.7 | 17.5 | 14 | 8.4 |
| 8 | Y | Cpd 4 | 100 | umol/kg | 6 | 0.3 | 12 | 6.4 | 3.8 | 1.9 |
| 9 | Y | Cpd 4 | 300 | umol/kg | 5.2 | 0.2 | 8.1 | 4.5 | 4.3 | 3.1 |
| 10 | Y | Cpd 4 | 10 | umol/kg | 7.8 | 0.4 | 20 | 8.7 | 6.4 | 2.9 |
| | | Colchicine | 0.3 | mpk | | | | | | |

### Example 10: Compound 3 and Compound 4 in Combination with Colchicine in a Therapeutic Model

Compounds 3 and 4 were tested, in the presence and absence of colchicine, at various doses, according to the protocol described in Example 2. 140 rats were used, divided into 14 groups of 10 animals, as follows:

| Group | Treatment | Dose | | ROA |
|---|---|---|---|---|
| 1 | Vehicle | N/A | | PO |
| 2 | Vehicle | N/A | | PO |
| 3 | Colchicine | 0.3 | mg/kg | SC |
| 4 | Colchicine | 0.5 | mg/kg | SC |
| 5 | Colchicine | 1.0 | mg/kg | SC |
| 6 | Cpd 4 | 10 | μmol/kg | PO |
| 7 | Cpd 4 | 30 | μmol/kg | PO |
| 8 | Cpd 4 | 100 | μmol/kg | PO |
| 9 | Cpd 4 | 10 | μmole/kg | PO |
| | Colchicine | 0.3 | mg/kg | SC |
| 10 | Cpd 3 | 3 | μmol/kg | PO |
| 11 | Cpd 3 | 10 | μmol/kg | PO |
| 12 | Cpd 3 | 30 | μmol/kg | PO |
| 13 | Cpd 3 | 100 | μmol/kg | PO |
| 14 | Cpd 3 | 10 | μmol/kg | PO |
| | Colchicine | 0.3 | mg/kg | SC |

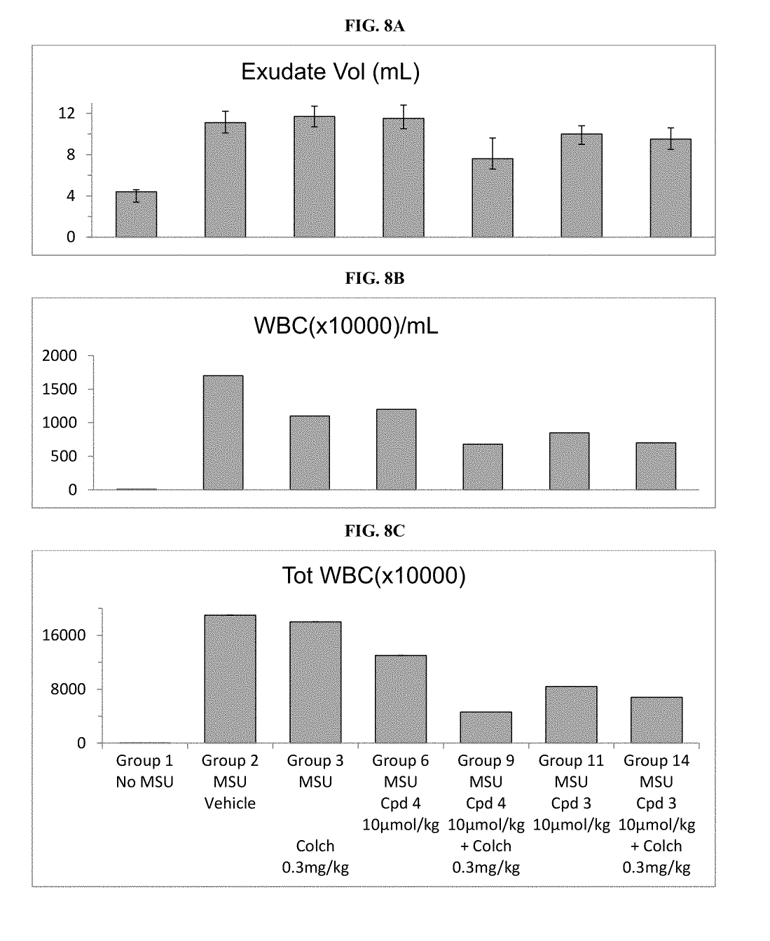

The results—average exudate volume (FIG. 8A), total white blood cell counts (FIG. 8B) and neutrophil counts (FIG. 8C) are provided in the table below and presented in FIGS. 8A-8C (Groups 1, 2, 3, 6, 9, 11 & 14).

| | | | | | Avg Exudate | | Avg To | Neutrophils | |
|---|---|---|---|---|---|---|---|---|---|
| Gp | MSU | Treatment | Dose | | Vol (mL) | SE | WBC (×10⁶) | (×10⁶) | SE |
| 1 | N | Vehicle | | | 4.4 | 0.2 | 0.38 | 26.9 | 8.1 |
| 2 | Y | Vehicle | | | 11.1 | 1.1 | 190 | 81.5 | 4.9 |
| 3 | Y | Colchicine | 0.3 | mpk | 11.7 | 1.0 | 180 | 71.5 | 3.8 |
| 4 | Y | Colchicine | 0.5 | mpk | 8.9 | 1.2 | 35 | 70.7 | 5.2 |
| 5 | Y | Colchicine | 1.0 | mpk | 7.3 | 1.1 | 9.8 | 25.9 | 7.5 |
| 6 | Y | Cpd 4 | 10 | μmol/kg | 11.5 | 1.3 | 130 | 68.4 | 7.6 |
| 7 | Y | Cpd 4 | 30 | μmol/kg | 8.2 | 1.6 | 25 | 60.4 | 7.2 |
| 8 | Y | Cpd 4 | 100 | μmol/kg | 7.0 | 1.6 | 18 | 26.2 | 6.4 |
| 9 | Y | Cpd 4 | 10 | μmole/kg | 7.6 | 2.0 | 46 | 61.5 | 6.4 |
| | | Colchicine | 0.3 | mg/kg | | | | | |
| 10 | Y | Cpd 3 | 3 | μmol/kg | 11.5 | 1.2 | 120 | 71.1 | 3.1 |
| 11 | Y | Cpd 3 | 10 | μmol/kg | 10.0 | 0.8 | 84 | 69.2 | 4.9 |
| 12 | Y | Cpd 3 | 30 | μmol/kg | 8.8 | 1.8 | 59 | 30.7 | 9.3 |
| 13 | Y | Cpd 3 | 100 | μmol/kg | 6.9 | 1.4 | 40 | 35.1 | 8.9 |
| 14 | Y | Cpd 3 | 10 | μmol/kg | 9.5 | 1.1 | 68 | 72.6 | 2.7 |
| | | Colchicine | 0.3 | mg/kg | | | | | |

### Example 11: Measurement of Interleukin-1β (IL-1β) and Myeloperoxidase (MPO) Plate Preparation

1. Dilute the Capture Antibody (to the working concentration stated in the product datasheet) in PBS without carrier protein. Immediately coat a 96-well microplate with 100 μL per well of the diluted Capture Antibody. Seal the plate and incubate overnight at room temperature.
2. Aspirate each well and wash with Wash Buffer, repeating the process two times for a total of three washes. Wash by filling each well with Wash Buffer (400 μL) using a squirt bottle, manifold dispenser, or autowasher. Complete removal of liquid at each step is essential for good performance. After the last wash, remove any remaining Wash Buffer by aspirating or by inverting the plate and blotting it against clean paper towels.
3. Block each well of the microplate as recommended in the product datasheet. Incubate at room temperature for a minimum of 1 hour.
4. Repeat the aspiration/wash as in step 2. The plates are now ready for sample addition.

Assay Procedure
1. Add 100 μL of sample or standards in Reagent Diluent, or an appropriate diluent, per well. Cover with an adhesive strip and incubate 2 hours at room temperature.
2. Repeat the aspiration/wash as in step 2 of Plate Preparation.
3. Add 100 μL of the Detection Antibody, diluted in Reagent Diluent (as recommended in the product datasheet), to each well. Cover with a new adhesive strip and incubate 2 hours at room temperature.
4. Repeat the aspiration/wash as in step 2 of Plate Preparation.
5. Add 100 μL of the working dilution of Streptavidin-HRP to each well. Cover the plate and incubate for 20 minutes at room temperature. Avoid placing the plate in direct light.
6. Repeat the aspiration/wash as in step 2.
7. Add 100 μL of Substrate Solution to each well. Incubate for 20 minutes at room temperature. Avoid placing the plate in direct light.
8. Add 50 μL of Stop Solution to each well. Gently tap the plate to ensure thorough mixing.
9. Determine the optical density of each well immediately, using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

### Example 12: Measurement of IL-1β and MPO in Exudate

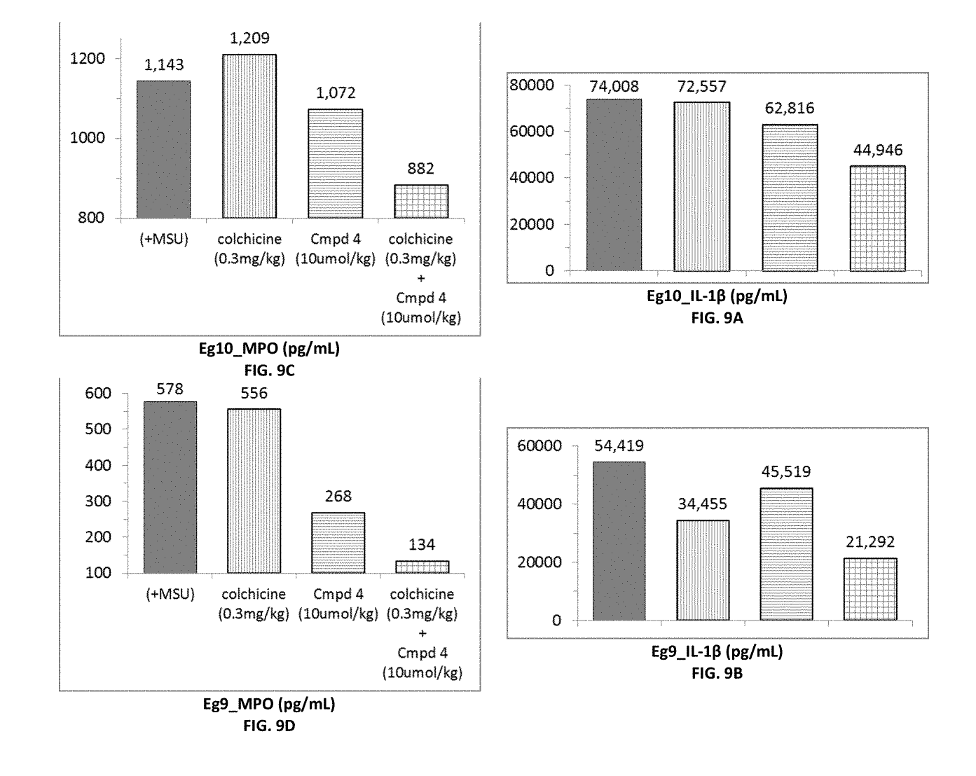

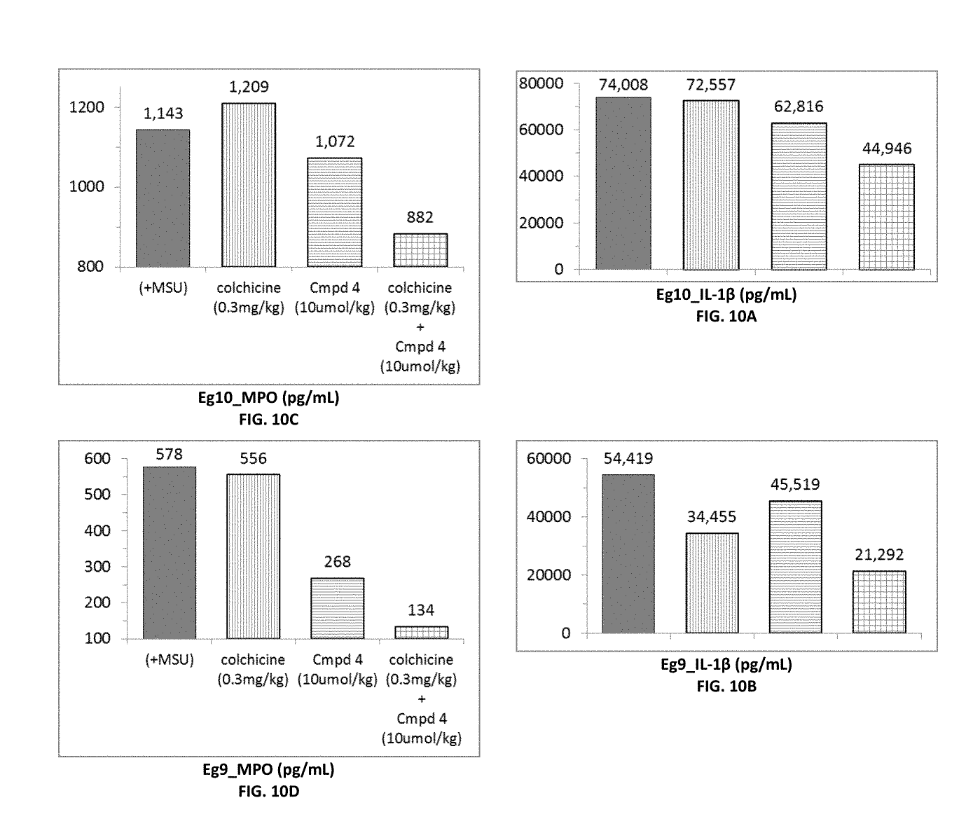

The exudate from select groups from examples 9 and 10 was examined for IL-1β and MPO content as described in example 11. The amounts (and Δ—differences from positive control) of IL-1β and MPO (pg/mL)—for groups 1, 2, 3, 6 and 10 (example 9) and groups 1, 2, 3, 6 and 9 (example 10) are provided in the table below and graphically presented in FIGS. 9A-9D and 10A-10D.

| Group | Treatment | Dose (mg/kg) | | ROA |
|---|---|---|---|---|
| 1 | Vehicle | 10 | ml/kg | PO |
| 2 | Vehicle | 10 | ml/kg | PO |
| 3 | Colchicine | 0.3 | mg/kg | SC |
| 6 | Cmpd 4 | 10 | μmol/kg | PO |

| Group | Treatment | Dose (mg/kg) | | ROA |
|---|---|---|---|---|
| 9 or 10 | Cmpd 4 | 10 | μmol/kg | PO |
| | Colchicine | 0.3 | mg/kg | SC |

| | | | | | | Group 10/9 |
|---|---|---|---|---|---|---|
| | | | | | | colchicine |
| Amount of | | Group 1 | Group 2 | Group 3 | Group 6 | (0.3 mg/kg) + |
| biomarker | | Control | Control | colchicine | Cmpd 4 | Cmpd 4 |
| (pg/mL) | | (−MSU) | (+MSU) | (0.3 mg/kg) | (10 μmol/kg) | (10 μmol/kg) |
| IL-1β | Eg 9 | 64 | 54,419 | 34,455 | 45,519 | 21,292 |
| | Δ | | | (−19,964) | (−8,900) | (−33,127) |
| | Eg 10 | 0 | 74,008 | 72,557 | 62,816 | 44,946 |
| | Δ | | | (−1,451) | (−11,192) | (−29,062) |
| MPO | Eg 9 | 2,214 | 577,573 | 555,723 | 267,983 | 134,120 |
| | Δ | | | (21,850) | (−309,590) | (−443,453) |
| | Eg 10 | 14,274 | 1,143,072 | 1,208,767 | 1,071,538 | 882,305 |
| | Δ | | | 65,695 | (71,534) | (−260,767) |

### Example 13: Cell Migration Assay (Ex Vivo Cultured Neutrophils)

### Example 13A: Chemotaxis Assay

Chemotaxis in Transwell Plates & CyQUANT Quantification

Chemotaxis was assayed in 96-well plates with mouse bone marrow-derived, ex vivo cultured neutrophils accordingly to the procedure set forth below.

Assay Procedure
1. Seed 1×10⁶ mature neutrophils per well in a 6-well plate (2×10⁵ cells/mL in 5 mL growth media)—1 well per drug concentration.
2. Add appropriate volume of drug or DMSO (control) to each well.
3. Incubate the plate for 30 min at 37° C./5% CO₂.
4. Harvest the cells by passage into separate 15 mL conical tubes on ice and centrifuge 5 min at 1250 rpm.
5. Resuspend the cells in serum- and phenol red-free IMDM at 1×10⁵ cells/50 μL.
6. Using a repeat pipettor, add 150 μL of media to bottom chambers of the transwell plate (layout shown below)
   i. Serum- and phenol red-free IMDM (background)
   ii. Serum- and phenol red-free IMDM+1% certified FBS (negative control)
   iii. Serum- and phenol red-free IMDM+1% certified FBS+100 ng/mL KC
7. Apply the upper chambers on the bottom plate and carefully add 50 μL of the cells into the appropriate wells.
8. Cover the plate and incubate at 37° C./5% CO₂ for 2 h.
9. At the end of the incubation time, thaw all the ingredients and prepare CyQUANT solution in dark—i.e., to prepare 15 mL of the solution, add 60 μL CyQUANT Direct Nucleic Acid Stain and 300 μL CyQUANT Direct Background Suppressor to 15 mL 1×HBSS.
10. At the end of the incubation time, carefully remove the upper chambers and add 150 μL CyQUANT solution to each bottom well using a repeat pipettor.
11. Incubate for 1 h at 37° C./5% CO₂.
12. At the end of the incubation time, set up a protocol for the plate reader—excitation/emission: 485/528 nm; and gain: 85-90.
13. After the incubation is complete, remove the lid, place the plate in plate reader and read the fluorescence signal.

| | | | | | Drug | | Drug | | Drug | |
|---|---|---|---|---|---|---|---|---|---|---|
| | | | | | Concentra- | | Concentra- | | Concentra- | |
| | | DMSO | | | tion 1 | | tion 2 | | tion 3 | |
| + cells | A | i | ii | iii | ii | iii | ii | iii | ii | iii |
| | B | i | ii | iii | ii | iii | ii | iii | ii | iii |
| | C | i | ii | iii | ii | iii | ii | iii | ii | iii |
| | D | i | ii | iii | ii | iii | ii | iii | ii | iii |
| | E | i | ii | iii | ii | iii | ii | iii | ii | iii |
| No cells | F | i | ii | iii | ii | iii | ii | iii | ii | iii |
| | G | i | ii | iii | ii | iii | ii | iii | ii | iii |
| | H | i | ii | iii | ii | iii | ii | iii | ii | iii |

i. Serum- and phenol red-free IMDM (background);
ii. Serum- and phenol red-free IMDM + 1% certified FBS (negative control)
iii. Serum- and phenol red-free IMDM + 1% certified FBS + 100 ng/mL KC

### Example 13B: Results

Three concentrations of compound 4 were tested and the approximate percentage responses compared to DMSO (the diluent/control) were as follows: 1 μM: ˜75%; 10 M: ˜40%, 100 μM: ˜35%. Even at the lowest concentration of 1 μM, compound 4 caused at least a 25% inhibition of neutrophil chemotaxis, or only 75% of the cells migrated as compared to the controls. Therefore, compound 4 demonstrated significant inhibition of migration in neutrophils. These results confirm a dose-dependent inhibitory response and efficacy of compound 4 even at low concentrations.

### Example 13C: Chemotaxis Assay

Chemotaxis is assayed in 96-well plates with mouse bone marrow-derived, ex vivo cultured neutrophils accordingly to the procedure set forth above in Example 13A with colchicine.

### Example 13D: Chemotaxis Assay

Chemotaxis is assayed in 96-well plates with mouse bone marrow-derived, ex vivo cultured neutrophils accordingly to the procedure set forth above in Example 13A with a combination of colchicine and compound 4.

### Example 14: Studies for the Treatment of Dermatological Disorders

### Example 14A: Mouse Models

The skin-humanized psoriasis and atopic dermatitis mouse models (described in J. of Invest. Dermatol. 2016; 136: 163-145) are used to demonstrate the efficacy of compound 4, alone and in combination with colchicine. Other mouse models include those induced by epicutaneous application of sensitizers, transgenic and knockout mice, and spontaneous mouse models of atopic dermatitis and psoriasis. (J. Invest. Dermatol. 2009; 129: 31-40. J. Invest. Dermatol. 2007; 127(6): 1292-1308.)

### Example 14B: Imiquimod-Induced Psoriasis Mouse Model for In Vivo Efficacy Screening

5% IMQ cream is applied on the skin and/or ear of mice daily for five days causing erythema, scaling, and skin thickening. Histologically, any epidermal changes such as the infiltration of inflammatory cells, as well as hyper- and parakeratosis, are noted in IMQ-treated skin. Topical application of the IMQ cream causes the enlargement of spleen and lymph nodes, and increased levels of cytokines such as IL-23, TNFα and IL-17 in the affected skin tissues. Compounds 4 is tested, in the presence and absence of colchicine, at various doses in the IMQ-induced psoriasis mouse model. As appropriate, the rats are dosed orally with compound 4 or vehicle (saline alone). As appropriate, the rats are injected subcutaneously with colchicine (1 mL/kg). As appropriate, the rats are dosed with compound 4 (oral administration) in combination with colchicine (injected subcutaneously).

### Example 14C: Clinical Trial for Treating Patients with Psoriasis with Compound 4 in Combination with Colchicine

The purpose of this study is to evaluate primarily the safety and tolerability and secondarily the efficacy of topically applied or orally administered combination of colchicine and compound 4 in patients with mild to severe psoriasis. Enrolled patients are healthy male and female patients aged 18-65 y.o. with clinical diagnosis of stable plaque psoriasis for >months affecting a maximum of 6% of BSA with a minimum of 0.5% BSA on each side of the body and a minimum of one plaque at least 2×2 cm on each side excluding elbow and knee. This study is a double-blind, randomized, placebo controlled study of patients, where the patients are treated for 28 days plus a 7 day follow-up in which the patients. During the treatment period, patients visit the study center weekly for safety, tolerability and efficacy assessment. On these study visit days, blood is drawn prior to that morning's application of topical or oral administration. Efficacy is assessed by PGA, target lesion assessment and BSA. Safety is assessed through vital signs, ECG, AEs and Plasma PK via Cmin, Cmax, Tmax and AUCo-t.

Primary Outcome Measures: Evaluate safety and tolerability of topically applied or orally administered combination of colchicine and compound 4, and to assess the pharmacokinetics based on plasma concentrations.

Secondary Outcome Measures: Obtain initial evaluation of efficacy of topically applied or orally administered combination of colchicine and compound 4 in patients with mild to moderate psoriasis.

1.-3. (canceled)

4. A method for treating or preventing a chemokine-mediated disease or disorder, an interleukin-1 (IL-1)-mediated disease or disorder and/or a myeloperoxidase (MPO)-mediated disease or disorder in a subject in need thereof, comprising administering to the subject, a therapeutically effective amount of a combination of:
(i) Colchicine, or a pharmaceutically acceptable salt thereof; and
(ii) a CXCR-2 inhibitor, or a pharmaceutically acceptable salt thereof, wherein the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide:

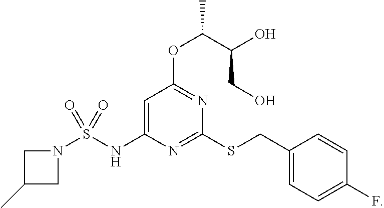 [SMILES: CC1CN(S(=O)(=O)Nc2cc(O[C@H](C)[C@@H](O)CO)nc(SCc3ccc(F)cc3)n2)C1]

5. The method of claim 4, wherein the combination is a synergistic combination.

6. The method of claim 4, wherein the IL-1-mediated disease or disorder is an inflammation-mediated disease or disorder, an autoimmune disease or disorder, a neutrophil-mediated disease or disorder, or a hematopoietic disease or disorder.

7.-8. (canceled)

9. The method of claim 6, wherein the inflammation-mediated disease or disorder is a neuroinflammatory disease or disorder, a dermatological disease or skin disorder, or pancreatitis.

10. (canceled)

11. The method of claim 9, wherein the neuroinflammatory disease or disorder is Alzheimer's Disease.

12. The method of claim 9, wherein the pancreatitis is acute pancreatitis, chronic pancreatitis, alcohol induced pancreatitis, gallstone induced pancreatitis, drug induced pancreatitis, auto-immune pancreatitis, procedure induced pancreatitis, or trauma induced pancreatitis, or any combination thereof.

13. The method of claim 9, wherein the dermatological disease or skin disorder is rosacea, eczema, acne, hidradenitis suppurativa, Palmo-Plantar Pustulosis, Generalized Pustular Psoriasis, Pyoderma Gangrenosum, Erosive Pustular Dermatosis of the Scalp, Sweet's Syndrome, Bowel-associated Dermatosis-arthritis Syndrome, Pustular Psoriasis, Acute Generalized Exanthematous Pustulosis, Keratoderma Blenorrhagicum, Sneddon-Wilkinson Disease, IgA Pemphigus, Amicrobial Pustulosis of the Folds, Infantile Acropustulosis, Transient Neonatal Pustulosis, Neutrophilic Eccrine Hidradenitis, Rheumatoid Neutrophilic Dermatitis, Neutrophilic Urticaria, Dermatitis Herpetiformis, Linear IgA disease (LAD), Inflammatory Epidermolysis Bullosa Aquisita, Alopecia Areata, Autoimmune Angioedema, Autoimmune progesterone dermatitis, Autoimmune urticaria, Bullous pemphigoid, Cicatricial pemphigoid, Dermatitis herpetiformis, Epidermolysis bullosa acquisita, Erythema nodosum, Gestational pemphigoid, Lichen planus, Lichen sclerosus, Morphea, Pemphigus vulgaris, Pityriasis lichenoides et varioliformis acuta, Mucha-Habermann disease, Vitiligo, or Neutrophilic Dermatosis of the Dorsal Hands, or any combination thereof.

14. The method of claim 6, wherein the autoimmune disease or disorder is Pustular Vasculitis, Small Vessel Vasculitis, Urticarial Vasculitis, Autoimmune urticaria, Medium Vessel Vasculitis, rheumatoid arthritis, Celiac disease, Graves' disease, Sjorgen syndrome, scleroderma, thyroiditis, myasthenia gravis, vasculitis, Addison's disease, autoimmune hepatitis, myocarditis, postmyocardial infarction syndrome, postpericardiotomy syndrome, subacute bacterial endocardititis, Anti-Glomerular Basement Membrane nephritis, Interstitial cystitis, lupus nephritis, systemic lupus, bullous systemic lupus erythmatosus, Primary biliary cirrhosis (PBC), Primary sclerosing cholangitis, Antisynthetase syndrome, Ord's thyroiditis, Autoimmune Oophoritis, Autoimmune orchitis, Autoimmune enteropathy, Chron's disease, microscopic colitis, ulcerative colitis, Antiphospholipid syndrome (APS), Aplastic anemia, Autoimmune hemolytic anemia, Autoimmune lymphoproliferative syndrome, Autoimmune neutropenia, or Autoimmune thrombocytopenic purpura.

15. The method of claim 6, wherein the hematopoietic disease or disorder is an anemia, a blood coagulation disorder, a blood platelet disorder, a blood protein disorder, erythroblastosis, hematologic neoplasm, hemoglobinopathies, a hemorrhagic disorder, a leukocyte disorder, methemoglobinemia, pancytopenia, polycythemia, preleukemia, sulfhemoglobinemia, or thrombophilia.

16. The method of claim 4, wherein the IL-1-mediated disease or disorder is a disease of the respiratory tract, a disease of the bones and/or joints, a skin disease, a disease of the gastrointestinal tract, a disease of central and/or peripheral nervous system, cancer, cystic fibrosis, a burn wound, a chronic skin ulcer, a reproductive disease, a re-perfusion injury, allograft rejection, atherosclerosis, Acquired Immunodeficiency Syndrome (AIDS), lupus erythematosus, systemic lupus erythematosus, Hashimoto's thyroiditis, diabetes mellitus type I, diabetes mellitus type II, nephrotic syndrome, eosinophilia fascitis, hyper IgE syndrome, lepromatous leprosy, idiopathic thrombocytopenia pupura, post-operative adhesions, sepsis, septic shock, Behcet's Disease, Still's Disease, Erythema Marginatum, Unclassified Periodic Fever Syndromes, Autoinflammatory Syndromes, or Erythema Elevatum Diutinum, or any combination thereof.

17.-19. (canceled)

20. The method of claim 6, wherein the inflammation-mediated disease or disorder is related to elevated CXCR-2 levels.

21.-30. (canceled)

31. A pharmaceutical composition comprising i) colchicine, or a pharmaceutically acceptable salt thereof; ii) a CXCR-2 inhibitor, or a pharmaceutically acceptable salt thereof; and iii) a pharmaceutically acceptable excipient, wherein the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide:

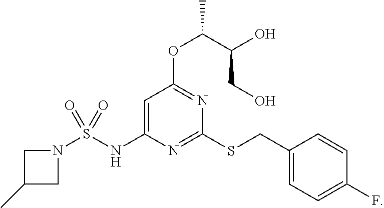 [SMILES: CC1CN(S(=O)(=O)Nc2cc(O[C@H](C)[C@@H](O)CO)nc(SCc3ccc(F)cc3)n2)C1]

32. A method for treating or preventing a chemokine-mediated disease or disorder, an interleukin-1 (IL-1)-mediated disease or disorder and/or a myeloperoxidase (MPO)-mediated disease or disorder in a subject in need thereof, comprising administering to a subject in need thereof, a therapeutically effective amount of the pharmaceutical composition of claim 31.

33. (canceled)
